(19) United States
(12) Patent Application Publication
(10) Pub. No.: US 2003/0070676 A1
(43) Pub. Date: Apr. 17, 2003

(54) Conduits having distal cage structure for maintaining collateral channels in tissue and related methods

(76) Inventors: Joel D. Cooper, St. Louis, MO (US); Thomas Keast, Sunnyvale, CA (US); Bryan Loomas, Los Gatos, CA (US); Ed Roschak, Mission Viejo, CA (US); Gary Kaplan, San Francisco, CA (US); Sandra Saenz, Mountain View, CA (US); Mike Collinson, Goleta, CA (US); Russ Redmond, Goleta, CA (US); Claude Vidal, Santa Barbara, CA (US); David Chandos, Lompoc, CA (US); Michael Biggs, Santa Clara, CA (US); Halil Karabey, San Jose, CA (US); Don Tanaka, Saratoga, CA (US); David Thompson, San Jose, CA (US)

Correspondence Address: MORRISON & FOERSTER LLP, 755 PAGE MILL RD, PALO ALTO, CA, 94304-1018, US

(21) Appl. No.: 10/235,240

(22) Filed: Sep. 4, 2002

Related U.S. Application Data
(63) Continuation-in-part of application No. 09/947,144, filed on Sep. 4, 2001.
(63) Continuation-in-part of application No. 09/633,651, filed on Aug. 7, 2000.
(60) Provisional application No. 60/317,338, filed on Sep. 4, 2001.
(60) Provisional application No. 60/334,642, filed on Nov. 29, 2001.
(60) Provisional application No. 60/367,436, filed on Mar. 20, 2002.
(60) Provisional application No. 60/374,022, filed on Apr. 19, 2002.
(60) Provisional application No. 60/387,163, filed on Jun. 7, 2002.
(60) Provisional application No. 60/269,130, filed on Feb. 14, 2001.
(60) Provisional application No. 60/147,528, filed on Aug. 5, 1999.
(60) Provisional application No. 60/176,141, filed on Jan. 14, 2000.

(51) Int. Cl. A61M 16/00

(52) U.S. Cl. 128/200.24

(57) ABSTRACT

Devices and related methods are directed to altering gaseous flow within a lung to improve the expiration cycle of, for instance, an individual having Chronic Obstructive Pulmonary Disease. More particularly, conduits maintain collateral openings or channels through the airway wall so that air is able to pass directly out of the lung tissue to facilitate both the exchange of oxygen ultimately into the blood and/or to decompress hyper-inflated lungs. The conduits include a center section with a passageway extending through the center section. The conduits further include a distal cage structure which has a passageway and at least one opening in fluid communication with the center section passageway. The medical kits disclosed herein are also directed to maintain collateral openings through airway walls.

### CROSS-REFERENCE TO RELATED APPLICATIONS

This application claims the benefit of U.S. Provisional Application No. 60/317,338, filed on Sep. 4, 2001; U.S. Provisional Application No. 60/334,642, filed on Nov. 29, 2001; U.S. Provisional Application No. 60/367,436, filed on Mar. 20, 2002; U.S. Provisional Application No. 60/374,022, filed on Apr. 19, 2002; and U.S. Provisional Application No. 60/387,163, filed on Jun. 7, 2002. This application is also a continuation in part of U.S. application Ser. No. 09/947,144, filed Sep. 4, 2001, which claims the benefit of U.S. Provisional Application No. 60/269,130, filed on Feb. 14, 2001, and U.S. application Ser. No. 09/947,144, filed Sep. 4, 2001, is a continuation in part of U.S. application Ser. No. 09/633,651, filed Aug. 7, 2000, which claims the benefit of U.S. Provisional Application No. 60/147,528, filed on Aug. 5, 1999, and U.S. Provisional Application No. 60/176,141, filed on Jan. 14, 2000. Each of the above referenced applications is incorporated herein by reference in its entirety.

### FIELD OF THE INVENTION

The invention is directed to conduits for altering gaseous flow within a lung to improve the expiration cycle of an individual, particularly individuals having Chronic Obstructive Pulmonary Disease. The conduits maintain collateral openings or channels through the airway wall so that air is able to pass directly out of the lung tissue to facilitate both the exchange of oxygen ultimately into the blood and/or to decompress hyper-inflated lungs. The conduits generally include a center section having a passageway for air to flow through and a distal cage structure having a passageway that is in fluid communication with the center section passageway. The invention is also directed to methods and medical kits for maintaining collateral openings through airway walls.

### BACKGROUND OF THE INVENTION

In 1995, the American Lung Association (ALA) estimated that between 15-16 million Americans suffered from chronic obstructive pulmonary disease (COPD) which includes diseases such as chronic bronchitis, emphysema, and some types of asthma. The ALA estimated that COPD was the fourth-ranking cause of death in the U.S. The ALA estimates that the rates of emphysema is 7.6 per thousand population, and the rate for chronic bronchitis is 55.7 per thousand population.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

To understand the detrimental effects of COPD, the workings of the lungs requires a cursory discussion. The primary function of the lungs is to permit the exchange of two gasses by removing carbon dioxide from arterial blood and replacing it with oxygen. Thus, to facilitate this exchange, the lungs provide a blood gas interface. The oxygen and carbon dioxide move between the gas (air) and blood by diffusion. This diffusion is possible since the blood is delivered to one side of the blood-gas interface via small blood vessels (capillaries). The capillaries are wrapped around numerous air sacs called alveoli which function as the blood-gas interface. A typical human lung contains about 300 million alveoli.

The air is brought to the other side of this blood-gas interface by a natural respiratory airway, hereafter referred to as a natural airway or airway, consisting of branching tubes which become narrower, shorter, and more numerous as they penetrate deeper into the lung. Specifically, the airway begins with the trachea which branches into the left and right bronchi which divide into lobar, then segmental bronchi. Ultimately, the branching continues down to the terminal bronchioles which lead to the alveoli. Plates of cartilage may be found as part of the walls throughout most of the airway from the trachea to the bronchi. The cartilage plates become less prevalent as the airways branch. Eventually, in the last generations of the bronchi, the cartilage plates are found only at the branching points. The bronchi and bronchioles may be distinguished as the bronchi lie proximal to the last plate of cartilage found along the airway, while the bronchiole lies distal to the last plate of cartilage. The bronchioles are the smallest airways that do not contain alveoli. The function of the bronchi and bronchioles is to provide conducting airways that lead air to and from the gas-blood interface. However, these conducting airways do not take part in gas exchange because they do not contain alveoli. Rather, the gas exchange takes place in the alveoli which are found in the distal-most end of the airways.

The mechanics of breathing include the lungs, the rib cage, the diaphragm and abdominal wall. During inspiration, inspiratory muscles contract increasing the volume of the chest cavity. As a result of the expansion of the chest cavity, the pleural pressure, the pressure within the chest cavity, becomes sub-atmospheric. Consequently, air flows into the lungs and the lungs expand. During unforced expiration, the inspiratory muscles relax and the lungs begin to recoil and reduce in size. The lungs recoil because they contain elastic fibers that allow for expansion, as the lungs inflate, and relaxation, as the lungs deflate, with each breath. This characteristic is called elastic recoil. The recoil of the lungs causes alveolar pressure to exceed atmospheric pressure causing air to flow out of the lungs and deflate the lungs. If the lungs' ability to recoil is damaged, the lungs cannot contract and reduce in size from their inflated state. As a result, the lungs cannot evacuate all of the inspired air.

In addition to elastic recoil, the lungs' elastic fibers also assist in keeping small airways open during the exhalation cycle. This effect is known as “tethering” of the airways. Such tethering is desirable since small airways do not contain cartilage that would otherwise provide structural rigidity for these airways. Without tethering, and in the absence of structural rigidity, the small airways collapse during exhalation and prevent air from exiting thereby trapping air within the lung.

Emphysema is characterized by irreversible biochemical destruction of the alveolar walls that contain the elastic fibers, called elastin, described above. The destruction of the alveolar walls results in a dual problem of reduction of elastic recoil and the loss of tethering of the airways. Unfortunately for the individual suffering from emphysema, these two problems combine to result in extreme hyperinflation (air trapping) of the lung and an inability of the person to exhale. In this situation, the individual will be debilitated since the lungs are unable to perform gas exchange at a satisfactory rate.

One further aspect of alveolar wall destruction is that the airflow between neighboring air sacs, known as collateral ventilation or collateral air flow, is markedly increased as when compared to a healthy lung. While alveolar wall destruction decreases resistance to collateral ventilation, the resulting increased collateral ventilation does not benefit the individual since air is still unable to flow into and out of the lungs. Hence, because this trapped air is rich in CO₂, it is of little or no benefit to the individual.

Chronic bronchitis is characterized by excessive mucus production in the bronchial tree. Usually there is a general increase in bulk (hypertrophy) of the large bronchi and chronic inflammatory changes in the small airways. Excessive amounts of mucus are found in the airways and semisolid plugs of this mucus may occlude some small bronchi. Also, the small airways are usually narrowed and show inflammatory changes.

Currently, although there is no cure for COPD, treatment includes bronchodilator drugs, and lung volume reduction surgery. The bronchodilator drugs relax and widen the air passages thereby reducing the residual volume and increasing gas flow permitting more oxygen to enter the lungs. Yet, bronchodilator drugs are only effective for a short period of time and require repeated application. Moreover, the bronchodilator drugs are only effective in a certain percentage of the population of those diagnosed with COPD. In some cases, patients suffering from COPD are given supplemental oxygen to assist in breathing. Unfortunately, aside from the impracticalities of needing to maintain and transport a source of oxygen for everyday activities, the oxygen is only partially functional and does not eliminate the effects of the COPD. Moreover, patients requiring a supplemental source of oxygen are usually never able to return to functioning without the oxygen.

Lung volume reduction surgery is a procedure which removes portions of the lung that are over-inflated. The improvement to the patient occurs as a portion of the lung that remains has relatively better elastic recoil which allows for reduced airway obstruction. The reduced lung volume also improves the efficiency of the respiratory muscles. However, lung volume reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung volume reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung volume reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous. The production and maintenance of collateral openings through an airway wall allows air to pass directly out of the lung tissue responsible for gas exchange. These collateral openings serve to decompress hyper inflated lungs and/or facilitate an exchange of oxygen into the blood.

Methods and devices for creating, and maintaining collateral channels are discussed in U.S. patent application Ser. No. 09/633,651, filed on Aug. 7, 2000; U.S. patent application Ser. Nos. 09/947,144, 09/946,706, and 09/947,126 all filed on Sep. 4, 2001; U.S. Provisional Application No. 60/317,338 filed on Sep. 4, 2001; U.S. Provisional Application No. 60/334,642 filed on Nov. 29, 2001; U.S. Provisional Application No. 60/367,436 filed on Mar. 20, 2002; and U.S. Provisional Application No. 60/374,022 filed on Apr. 19, 2002 each of which is incorporated by reference herein in its entirety.

Events that may arise when a device is implanted in a surgically-created channel in a lung is that the device can be ejected, filled in with tissue, or otherwise rendered ineffective as the wound heals. It is desirable to provide a device which is capable of providing long-term patency of surgically-created channels in the lung and, in particular, to provide a device which is less susceptible to the above mentioned events.

### SUMMARY OF THE INVENTION

This invention relates to devices and methods for altering gaseous flow in a lung. The invention includes a conduit for maintaining the patency of an opening in tissue. In one variation of the present invention, a conduit comprises a center section having a first end, a second end and a center-section passageway extending from the first end to the second end. The conduit further comprises a plurality of first extension members extending from the first end. The first extension members are outwardly deflectable about the first end of the center section. The conduit further comprises a cage structure adjacent to the second end of the center section. The cage structure has at least one opening and a cage passageway in fluid communication with the center-section passageway.

The cage structure may connect directly to the second end of the center section or it may connect to the center section via a plurality of deflectable distal extension members which are joined to the second end of the center section. The cage may be formed of a single element or alternatively, the cage may be formed of a plurality of cage segments or members. Also, portions of the conduit may be coaxially surrounded with a tissue barrier or membrane to prevent tissue growth into the passageway of the conduit.

The conduit may have an undeployed state for facilitating delivery of the conduit to a target site and a deployed state, different than the undeployed state, for maintaining the patency of a channel in an airway wall. In this variation of the present invention the conduit comprises a radially expandable frame having a proximal section, a center section and a distal section. The proximal section comprises a plurality of proximal extension members. The center section comprises a first end at which the plurality of proximal extension members are attached. The center section further includes a second end and a center-section passage extending from the first end to the second end. The distal section of the frame comprises a cage having at least one opening and a cage passage which is in fluid communication with the center-section passage. When the conduit is in the undeployed state the proximal section, the center section, and the distal section have a reduced profile. When the conduit is in the deployed state, the plurality of extension members deflect outward forming a non-zero angle with an axis of the center-section passage, and the cage has an expanded profile greater than that of the cage when the conduit is in the undeployed state. Additionally, a biocompatible coating may coaxially surround at least a portion of the frame.

In another variation of the present invention a conduit comprises a center section having a proximal end, a distal end, and a passage within the center section extending between the ends. The conduit further comprises a plurality of extension members with at least one proximal extension member and at least one distal extension member. The proximal extension members have a fixed end attached to the proximal end of the center section and the distal extension members have a fixed end attached to the distal end of the center section. Also, each of the proximal and distal extension members have a free end being moveable such that the extension members may rotate about each of the ends of the center section to retain tissue between the extension members. The conduit further includes a cage adjacent to the distal end of the center section. The cage has at least one opening and a passage in fluid communication with the center section passage.

A method for maintaining the patency of a channel in lung tissue comprises deploying a medical device in the channel wherein the medical device has a passageway extending from one open end to a second open end. The medical device may be a conduit as recited herein. Also, the deploying step may be carried out using a balloon catheter having an inflatable member. The method may also comprise delivering a bioactive substance to the tissue. The bioactive substance may be a coating on the conduit. Additionally, the substance may be delivered by a delivery catheter prior to deploying the conduit.

A kit comprises a conduit as recited herein. The kit additionally comprises a deployment catheter to deploy the conduit. The deployment catheter may be a balloon catheter. The kit may further comprise an instrument for creating holes in an airway wall. The instrument may also have the capability to detect blood vessels when creating channels in the airway wall. The kit may further comprise a guidewire.

### BRIEF DESCRIPTION OF THE DRAWINGS

FIGS. 1A-1C illustrate various states of the natural airways and the blood-gas interface.

FIGS. 1D and 1E illustrate a schematic of a lung having conduits deployed in channels to alter airflow through the lung.

FIG. 2A illustrates a planar view of a surface of a variation of a conduit.

FIG. 2B illustrates a perspective view of the conduit of FIG. 2A in an un-deployed state.

FIG. 2C illustrates a perspective view of the conduit of FIG. 2A in a deployed state.

FIG. 3 illustrates an unexpanded planar view of a surface of a variation of a conduit in which cage members extend perpendicularly to distal extension members prior to deployment of the device.

FIGS. 4 and 5 illustrate planar views of variations of conduits.

FIG. 6 illustrates a planar view of a variation of a conduit wherein the proximal and distal extension members are in an alternating pattern.

FIG. 7A illustrates a planar view of a variation of a conduit of the present invention wherein the cage is attached to distal extension members.

FIG. 7B illustrates a perspective view of the conduit of FIG. 7A in a deployed state.

FIG. 8A illustrates a planar view of a variation of a conduit wherein the distal extension member is non-planar.

FIG. 8B illustrates a side view of the conduit of FIG. 8A.

FIG. 8C illustrates a perspective view of the conduit of FIG. 8B.

FIG. 9A illustrates a planar view of a variation of a conduit.

FIG. 9B illustrates a perspective view of the conduit of FIG. 9A in an un-deployed state.

FIG. 9C illustrates a perspective view of the conduit of FIG. 9A in a deployed state.

FIG. 9D illustrates a perspective view of another conduit in a deployed state.

FIG. 9E illustrates a side view of a conduit in an un-deployed state.

FIG. 9F illustrates a side view of the conduit of FIG. 9E shown in a deployed state.

FIG. 9G illustrates a front view of the conduit shown in FIG. 9F.

FIG. 9H is a cylindrical projection of the undeployed conduit shown in FIG. 9E.

FIG. 9I illustrates a side view of another conduit in an undeployed state.

FIG. 9J illustrates a side view of the conduit of FIG. 9I in a deployed state.

FIG. 9K is a cylindrical projection of the undeployed conduit shown in FIG. 9I.

FIGS. 9L-9P illustrate variations of conduits.

FIGS. 10A-10D illustrate a variation of the conduit having length-increasing portions on the cage members of the conduit.

FIG. 11 illustrates a cross sectional view of a variation of a conduit having an inner covering.

FIGS. 12A-12B illustrate views of a conduit having a filler material between openings in ribs.

FIGS. 13A-13C illustrate views of a conduit having reduced thickness or weakened sections.

FIG. 14A illustrates a variation of a conduit having a tissue barrier.

FIG. 14B illustrates a side view of another conduit having a tissue barrier.

FIG. 14C is a front view of the conduit shown in FIG. 14B.

FIG. 14D illustrates a conduit positioned in a channel created in a tissue wall.

FIG. 14E is a cross sectional view of the conduit shown in FIG. 14B taken along line 14E-14E.

FIGS. 14F-14K illustrate additional variations of conduits.

FIG. 15A illustrates a perspective view of another conduit.

FIG. 15B illustrates a side view of the conduit of FIG. 15A.

FIGS. 15C-15D illustrate planar views of a surface of the conduit shown in FIG. 15A.

FIGS. 15E and 15F illustrate a side view of the conduit of FIG. 15A prior to deployment of the proximal extension members and cage members.

FIG. 16A illustrates a planar view of a surface of a variation of a conduit.

FIG. 16B illustrates a perspective view of the conduit of FIG. 16A in a deployed state.

FIG. 16C illustrates a planar view of another variation of a conduit.

FIG. 16D illustrates a side view of the conduit shown in FIG. 16C in a deployed configuration.

FIG. 16E illustrates a side view of another conduit having a tissue barrier and a visualization marker.

FIG. 17A illustrates a side view of another conduit.

FIG. 17B illustrates a side view of the conduit shown in FIG. 17A after the conduit is deployed.

FIG. 17C illustrates a front view of the conduit shown in FIG. 17B.

FIGS. 17D-17E illustrate a variation of a conduit where the conduit comprises a wire or mesh pattern.

FIG. 17F illustrates another variation of a conduit.

FIGS. 18A-18F illustrate a method for deployment of a conduit.

FIGS. 19A-19C illustrate the deployment of a conduit.

FIGS. 20A-20B illustrate the deployment of a conduit.

FIGS. 21A-21C illustrate the deployment of a conduit using a balloon catheter.

FIG. 21D illustrates another variation of a balloon catheter which may be used to deploy a conduit.

FIGS. 22A-22D illustrate another variation of a deployment catheter which may be used to deploy conduits.

FIGS. 23A-23C illustrate the use of a guide member in assisting the placement of a conduit.

FIG. 24 illustrates a variation of a conduit having a one-way valve.

FIGS. 25A-25B illustrate a method for deploying a conduit at an angle.

### DETAILED DESCRIPTION OF THE INVENTION

Described herein are devices (and methods) for improving the gas exchange in the lung. In particular, a conduit is described which serves to maintain collateral openings or channels through an airway wall so that air is able to pass directly out of the lung tissue and into the airways. This facilitates exchange of oxygen into the blood and decompresses hyper inflated lungs.

By “channel” it is meant to include, but not be limited to, any opening, hole, slit, channel or passage created in the airway wall. The channel may be created in tissue having a discrete wall thickness and the channel may extend all the way through the wall. Also, a channel may extend through lung tissue which does not have well defined boundaries such as, for example, parenchymal tissue.

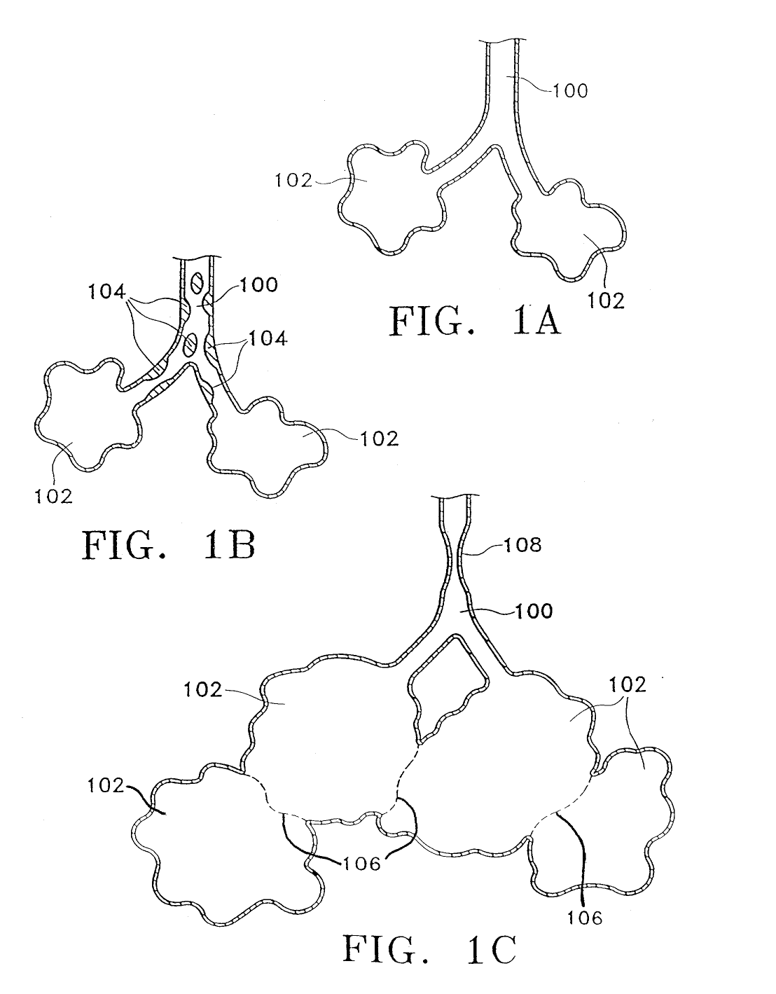

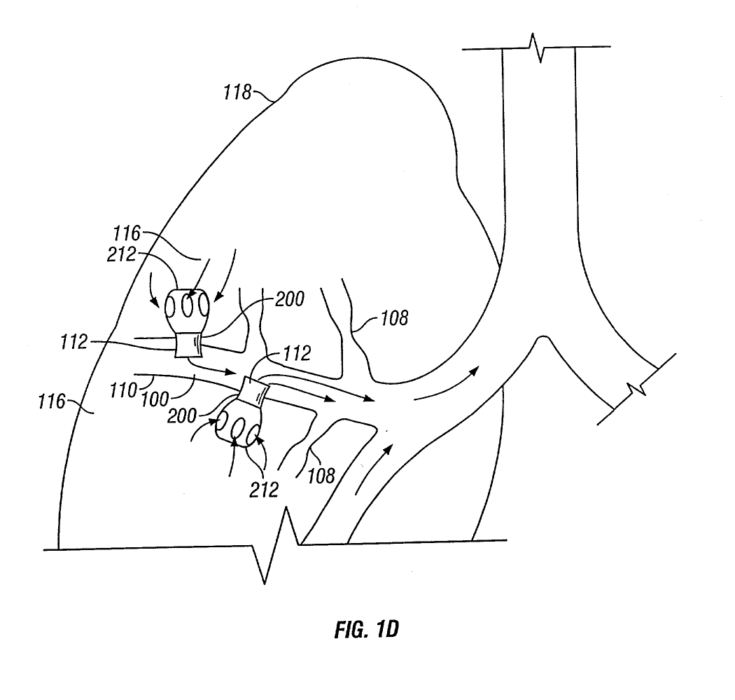

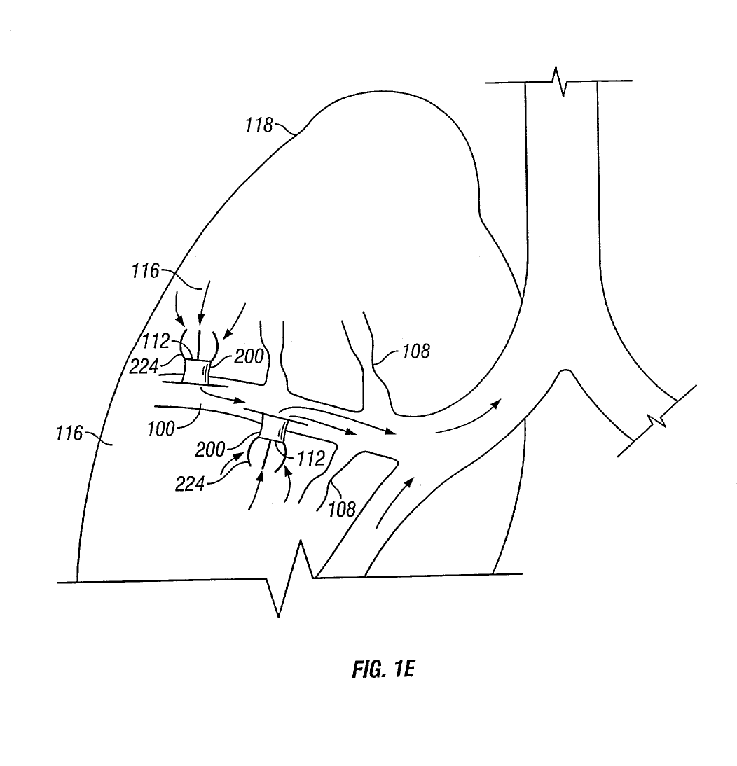

As stated above, the conduits described herein may improve airflow through an airway in the lung. Simplified illustrations of various states of a natural airway and a blood gas interface found at a distal end of those airways are provided in FIGS. 1A-1C. FIG. 1A shows a natural airway 100 which eventually branches to a blood gas interface 102. FIG. 1B illustrates an airway 100 and blood gas interface 102 in an individual having COPD. The obstructions 104 impair the passage of gas between the airways 100 and the interface 102. FIG. 1C illustrates a portion of an emphysematous lung where the blood gas interface 102 expands due to the loss of the interface walls 106 which have deteriorated due to, for example, a bio-chemical breakdown of the walls 106. Also depicted is a constriction 108 of the airway 100. It is generally understood that there is usually a combination of the phenomena depicted in FIGS. 1A-1C. Often, the states of the lung depicted in FIGS. 1B and 1C may be found in the same lung.

FIGS. 1D and 1E schematically illustrate airflow in a lung 118 when conduits 200 are placed in collateral channels 112. As shown, collateral channels 112 (located in an airway wall) place lung tissue 116 in fluid communication with airways 100 allowing air to pass directly out of the airways 100 avoiding constricted airways 108 which may ordinarily prevent air from exiting the lung tissue 116. The conduits shown in FIGS. 1D and 1E have cage structures 212, 224 respectively which serve to separate parenchymal tissue, prevent occlusion of the passageway, and improve air-flow through the conduit/collateral channel. The cage or basket structures may vary widely in shape and construction as will be described herein.

Also, while the invention is not limited to the number of collateral channels which may be created, it is preferable that 1 or 2 channels are placed per lobe of the lung. For example, the preferred number of channels is 2-12 channels per individual patient. However, as stated above, the invention includes the creation of any number of collateral channels in the lung. This number may vary on a case by case basis. For instance, in some cases an emphysematous lung may require 3 or more collateral channels in one or more lobes of the lung.

In the following explanation of figures, similar numerals may represent similar features for the different variations of the invention.

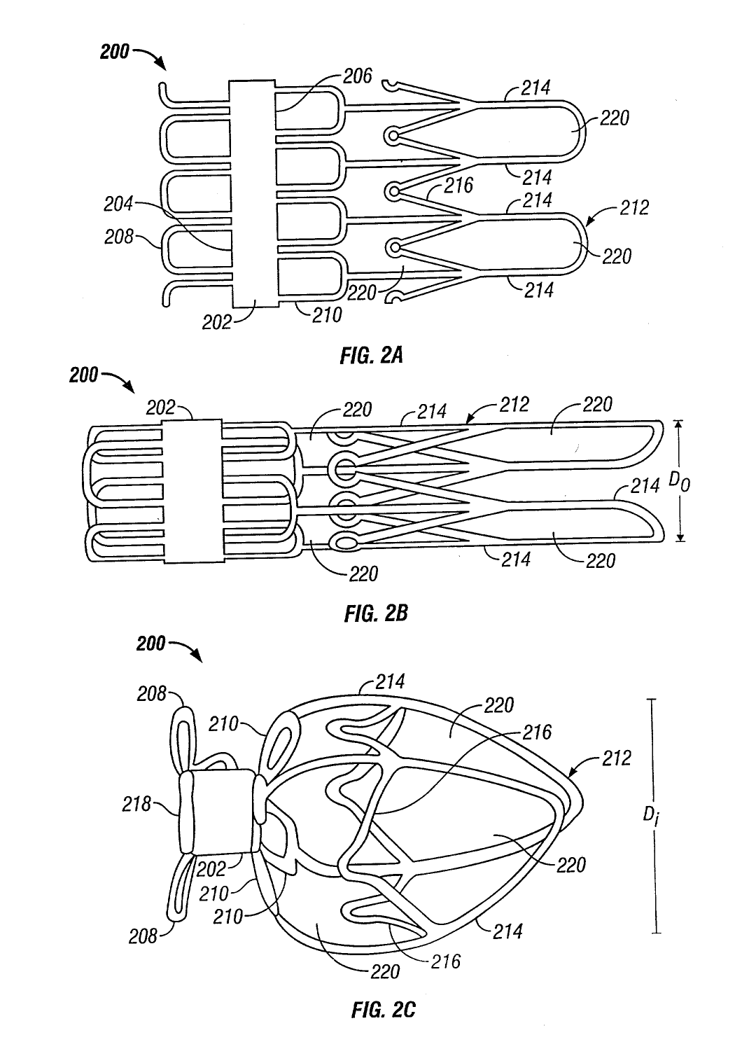

FIG. 2A illustrates a planar view of a surface of a variation of a conduit 200. For purposes of illustration, the conduit 200 depicted in FIG. 2A is shown as though the conduit 200 were longitudinally cut and opened. FIGS. 2B and 2C show the device in pre-deployed and deployed positions as discussed below. As illustrated, the conduit 200 comprises a center section 202, having proximal 204 and distal 206 ends. Although not illustrated in this figure, the center section 202 will define a passage which extends between its ends 204, 206. The conduit 200 also comprises at least one proximal extension member 208 at a proximal 204 end of the center section 202 and at least one distal extension member 210 at a distal 206 end of the center section 202. Although the conduit 200 depicted in the illustration contains 4 proximal and distal extension members 208, 210, the invention is not limited as such.

The conduits 200 of the present invention are not limited to any particular number of extension members. The number of proximal extension members may differ from the number of distal extension members for a particular conduit. The extension members will be selected such that they contain a fixed end that is attached to the respective end of the center section, and a movable (or free) end that is moveable such that the extension member is able to rotate or bend about the end of the center section. When the extension members rotate about the center section of the conduit, they are able to retain tissue therebetween thus preventing significant migration of the conduit. Accordingly, one function of the extension members is that they prevent migration of the inventive conduit from its deployed position within a collateral channel. The extension members may have openings to permit tissue ingrowth for improved retention within the lung. The opening may be used to anchor a tissue barrier that is located over a portion of the conduit. Alternatively, the extension members may be solid.

FIGS. 2A-2C illustrate a variation of a conduit 200 as having a cage 212. The cage 212 will define a passage (not shown) that is in fluid communication with a passage of the center section (not shown). This permits airflow through the conduit 200 in accordance with one of the benefits of the invention disclosed herein. In this variation, the cage 212 comprises a plurality of cage members 214. In use, the conduit 200 may be deployed with the distal 206 or cage side towards the parenchymal tissue while the proximal side 204 remains adjacent or in the airway.

Variations of the invention may include conduits 200 having expandable cages 212. Expansion of the cage 212 in the parenchymal tissue permits an increased surface area within the parenchyma to allow for improved air flow.

The presence of the cage may prevent flaps or portions of the parenchymal tissue from obstructing the passage of the center section. The cage may be effective immediately/dynamically as well as over time as the tissue heals.

Accordingly, in an unexpanded state, a portion of the cage 212 defines a first diameter and once expanded, the same portion of the cage 212 defines a second increased diameter. It is noted that the term diameter is not intended to limit the cage 212 to a cylinder or other constant diameter structure. Instead, the cage 212 may start with a constant diameter and is then expanded to have either a constant diameter, or a varying diameter (e.g., conical shape, rectangular shape, basket, other non-constant-diameter shape, or an asymmetrical shape.) Additionally, the center section 202 may or may not be expandable. In those cases where the center section 202 is expandable, once expanded, the diameter may be constant or vary (e.g., conical, hour-glass-shaped, hemi-toroidal, or other.)

Additionally, in some designs, one portion of the conduit may be radially expandable and another portion may not be radially expandable. For example, the center section may be designed as a hollow tubular member that is unexpandable. An expandable cage may be joined to the center section. Accordingly, a conduit may have various sections some of which are expandable and others which are not expandable.

The first diameter of the cage may be selected such that the conduit is small enough to fit within an airway, a Bronchoscope or another type of delivery instrument. The second diameter of the cage usually refers to the diameter of the cage after deployment. The relationship of the second diameter to the first diameter may be related as a ratio ranging from 1:1 to 4:1 or perhaps, 2:1 to 4:1. In the unexpanded state, the first diameter of the cage may range between 0.5 mm and 3 mm (including any range therebetween.) In the expanded state, the second diameter of the cage may range between 3 mm and 10 mm (including any range therebetween).

The axial length of the center section or passageway may be relatively short. In some cases, the center-section passageway's length is about equal to the width of a wire-segment or rib. The center section serves as a bridge or junction for the extension members and it is not required to be long. The axial length of the center-section passageway may therefore be less than 6 mm, less than 1 mm and even approach 0 mm. In one example, the length of the center section is less than twice the square root of a cross sectional area of the center section. However, the center section may also have passageways which have lengths greater than 1 mm.

The cage of the inventive conduit may have an axial length between 2 mm and 20 mm. The axial length will be measured along an axis of the passage of the conduit.

Cage 212 may be formed from an ordinary wire mesh that functions to keep parenchymal tissue separated to increase airflow through the conduit 200 passage. However, in the variation depicted in FIG. 2A, the cage 212 comprises a plurality of cage members 214 each of which is connected to a distal extension member 210. However, as will be discussed further below, it is contemplated that the invention also includes variations where cage members connect directly to the distal 206 end of the center section 202.

As shown in FIGS. 2A-2C, the cage 212 may comprise a plurality of control segments 216 which interconnect at least two cage members 214 (e.g., adjacent cage members). However, it is contemplated that variations of the inventive conduit may be designed without such control segments. One of the functions of the control segments 216 includes controlling the diameter of the cage 212 during expansion and/or prevention of over-expansion of the cage 212. Furthermore, the control segments described herein may be used to assist the conduit (or sections of the conduit) in achieving a desired profile upon expansion or deployment of the conduit. For example, the control segments may limit the expanded diameter to varying degrees along the axial length of the conduit.

FIG. 2A also illustrates the cage 212 as having openings 220. In this variation, the openings 220 of the cage 212 are defined by the cage members 214.

FIG. 2B illustrates a three-dimensional view of the conduit 200 of FIG. 2A, wherein the conduit 200 is in an un-deployed state. FIG. 2C illustrates a three-dimensional view of a conduit 200 of FIG. 2B after deployment of the conduit 200. The cage is in an expanded state in FIG. 2C. As illustrated, the proximal and distal extension members 208, 210 pivot (or rotate radially from a longitudinal axis of the device) around the center section 202 such that the extension members 208, 210 hold the conduit within a collateral channel. More specifically, tissue may be retained between extension members 208, 210 on the outer perimeter of the center section 202.

The conduit of the present invention may also be adapted to have a cage that is flexible. For instance, in the conduit 200 illustrated in FIG. 2B, cage 212 may be flexible so as to bend and flex when it contacts an object such as lung parenchyma, blood vessels, or adjacent airways, etc. Such flexibility may be achieved by making the cage members 214 (and control segments 216) sufficiently thin, or by designing them to have a sufficiently low elastic modulus. Such a flexible cage may prevent formation of scar tissue or erosion of adjoining tissues.

To facilitate deployment of the conduit of the present invention, the conduits described herein may have proximal extension members, distal extension members, and cages which are adapted to take a pre-determined form. For example, as will be discussed further herein, the conduit could be designed to have weakened sections at specific transition points to facilitate the conduit taking a desired shape.

The conduit of the present invention may also have a pre-set shape that is assumed upon deployment within a collateral channel. In such a case, the conduit may have a pre-deployment shape, either by being restrained (e.g., if the conduit is formed from a metal such as Nitinol and is elastically restrained into the pre-deployment shape), or by other measures that require actuation (e.g., as is the case with a shape-memory alloy which assumes a shape upon reaching a certain temperature.)

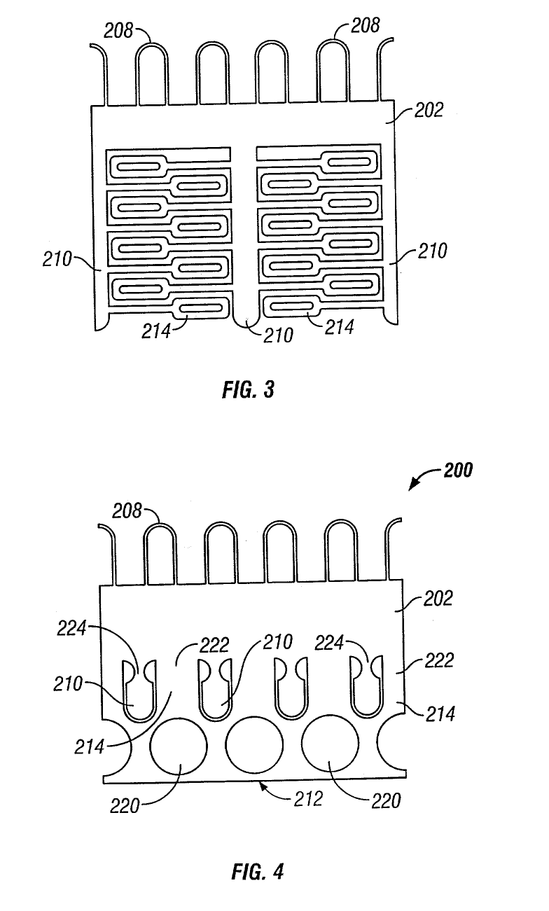

FIG. 3 illustrates an un-expanded planar view of a conduit 200 having a center section 202. The conduit 200 includes a plurality of proximal extension members 208 on a side of the center section 202. In the variation depicted in FIG. 3, the conduit 200 contains two distal extension members 210 from which a plurality of cage members 214 extend. As shown, the cage members 214 may contain openings to facilitate airflow, or, the cage members may be solid.

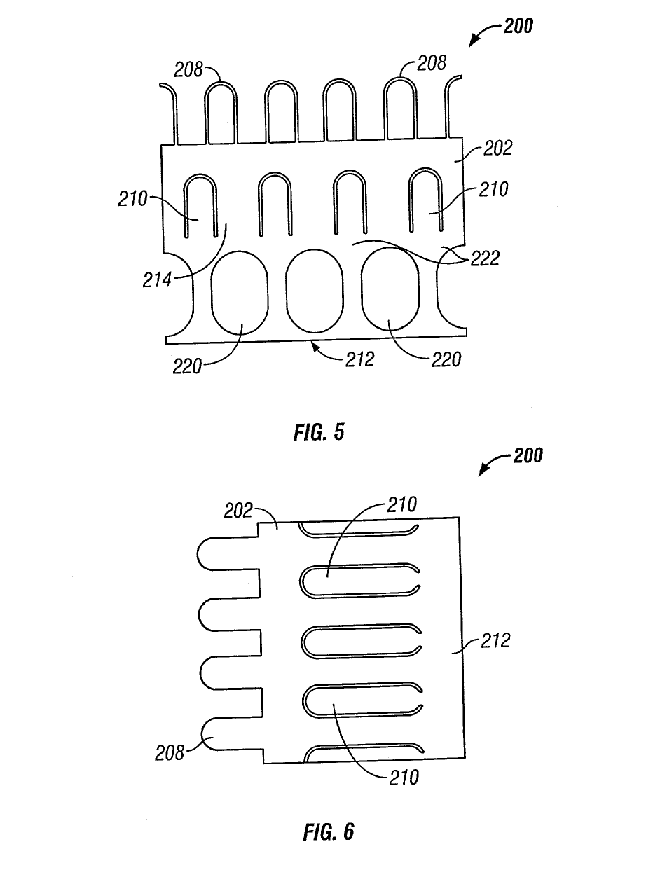

FIGS. 4 and 5 illustrate additional variations of conduits 200 in which the cage 212 is attached to a center section 202 of the conduit 200. In these variations, the center section 202 of the conduit 200 is located between proximal and distal extension members 208, 210. A proximal end 222 of the cage 212 is attached to the center section 202 of the conduit 200 via cage members 214 that extend between the distal extension members 210. The cage 212 may contain openings 220. The openings 220 may have a circular or oval geometries as illustrated. However, it is contemplated that the openings 220 are not limited to the particular geometry illustrated nor are the openings limited to the placement shown. For example, a conduit 200 could contain openings towards a distal end of the cage (as shown) and/or towards a proximal end of the cage (as discussed below). Furthermore, as illustrated in FIG. 4, the extension members may have reduced cross sectional areas 224 which increase the ability of the extension member to rotate about the end of the center section 202. FIG. 5 illustrates a variation of a conduit 200 in which the extension members extend in similar directions. Note that in this variation, the center section 202 will contain openings as the distal extension members 210 rotate away from the conduit 200. While not necessary, the center sections of conduits may be solid, e.g., to prevent tissue in-growth. Accordingly, a liner (not shown) could be placed in the passage of any center section that contains openings.

FIG. 6 illustrates another variation of a conduit 200 in which the proximal extension members 208 and distal extension members 210 are in an alternating pattern as opposed to an in-line pattern.

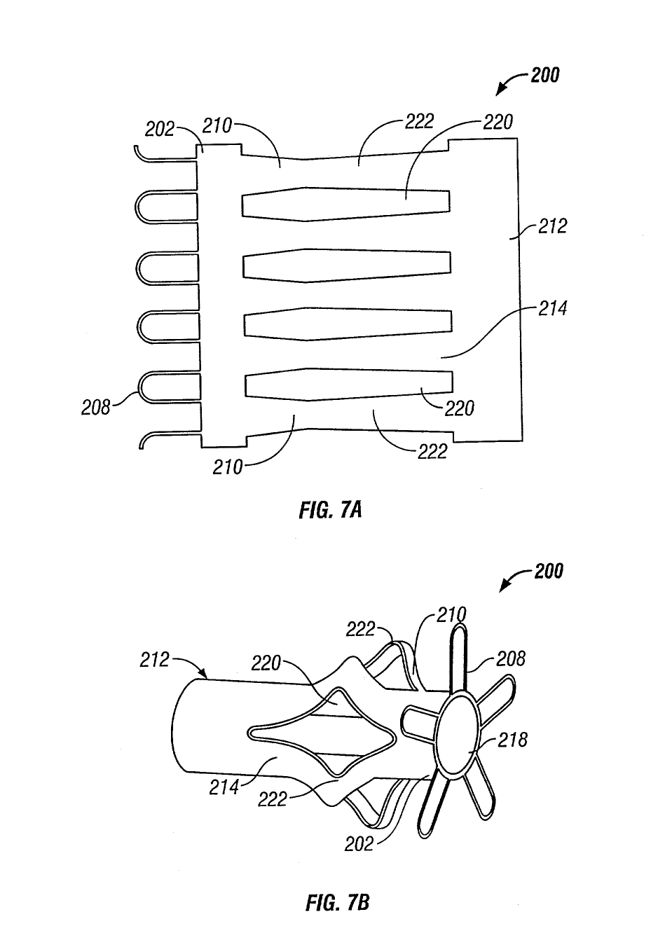

FIG. 7A illustrates another variation of an inventive conduit 200 in which a proximal end 222 of cage members 214 are attached to a free (or movable) end of the distal extension members 210. Accordingly, as the distal extension members 210 rotate about the center section 202 of the conduit 200, the cage 212 expands via expansion of the cage members 214. As illustrated, the conduit 200 may optionally have openings 220 between adjacent cage members 214. Although not illustrated, but as discussed above, the conduit may have openings on a distal portion of the cage 212 as well.

FIG. 7B illustrates a perspective view of the conduit 200 of FIG. 7A in a deployed position. As shown, the conduit 200, includes proximal extension members 208 and distal extension members 210 which are rotated about a center section 202 of the conduit 200. The cage 212 includes cage members 214 which have a proximal end 222 that is attached to a free end of the distal extension member 210. The cage 212 is shown to be in an expanded position and contains openings 220 which are intended to separate parenchymal tissue and allow for improved airflow through a passage 218 of the conduit 200. Additionally, although not shown in FIG. 7B, the passage 218 may extend through the conduit 200 and exit from a distal end of the cage 212. As discussed above, the cage 212 may optionally have additional openings on a distal end to further increase the airflow through the conduit passage 218.

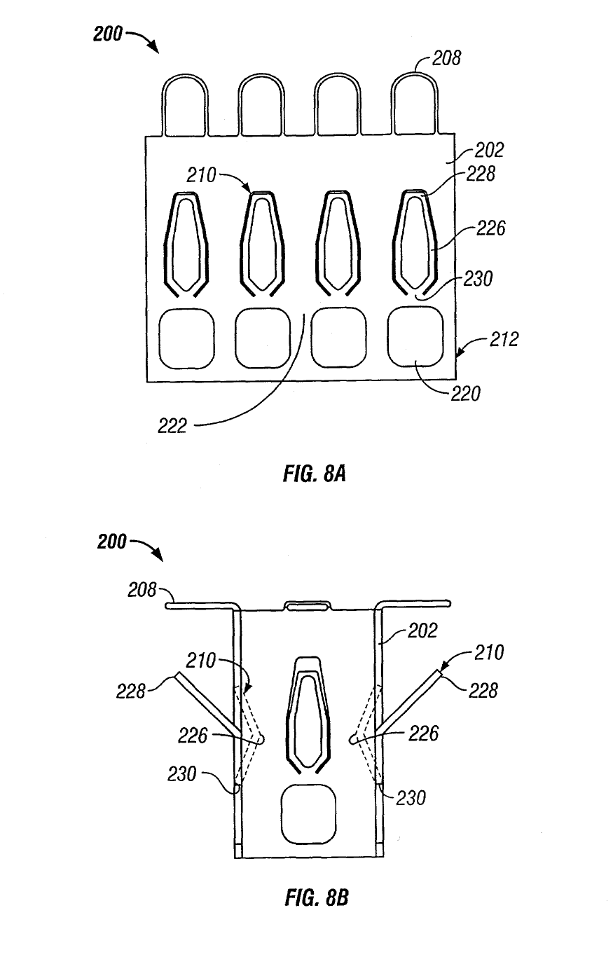

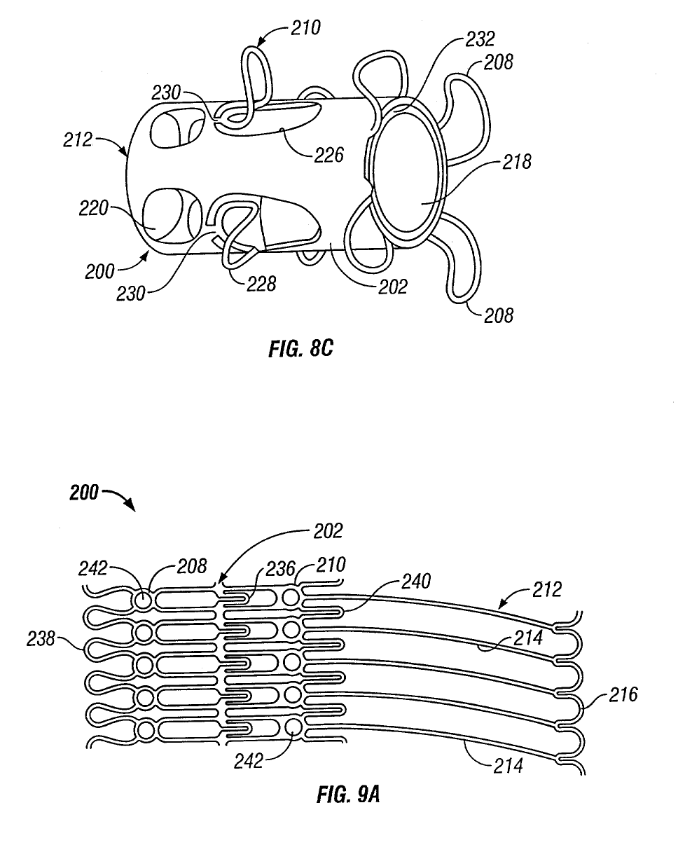

FIG. 8A illustrates another variation of an inventive conduit 200 having proximal and distal extension members 208, 210 and a cage 212 having a proximal end 222 attached to an end of a center section 202 of the conduit 200. The cage 212 also contains a number of openings 220 at a distal end thereof. The conduit 200 of FIG. 8A contains distal extension members 210 having a central portion 226 located between a free end 228 and a fixed end 230 of the extension member 210 where the free end 228 of the extension member 210 is non-planar with a plane defined by both the central portion 226 and the fixed end 230 of the extension member 210.

FIG. 8B provides a cross-sectional view of the conduit 200 of FIG. 8A and illustrates the central portion 226 of the extension member 210 as being non-planar with a plane defined by both the central portion 226 and the fixed end 230 of the extension member 210. Accordingly, as the conduit 200 is deployed, the central portion 226 is moved in an outward direction and aligns with the center section 202 of the conduit 200 causing the free end 228 of the extension member 210 to extend radially outward from the conduit 200. As a result, tissue may be secured between the free end 228 of the distal extension member 210 and the proximal extension member 208.

FIG. 8C illustrates a perspective view of the conduit 200 of FIG. 8B. As illustrated, the conduit 200 includes a number of proximal extension members 208 rotated about a center section 202 which contains a passage 218. Each distal extension member 210 contains a central portion 226 that is non-planar with a plane defined by the central portion 226 and the fixed end 230 of the extension member 210. As discussed above, as the central portion 226 is moved in an outward direction and aligns with the center section 202 of the conduit 200, the free end 228 of the extension member 210 extends radially outward from the conduit 200. As demonstrated by this variation, some conduits may be configured such that openings in the conduit 200 are placed within the collateral channel. Accordingly, a barrier layer or an inner covering 232 may be placed within the passage 218 of the center section 202 to prevent tissue in-growth. The inner covering 232 may be a tube or coating. Furthermore, the inner covering 232 may be sufficiently flexible such that the distal extension members 210 may deflect the inner covering 232 prior to deployment of the extension members 210.

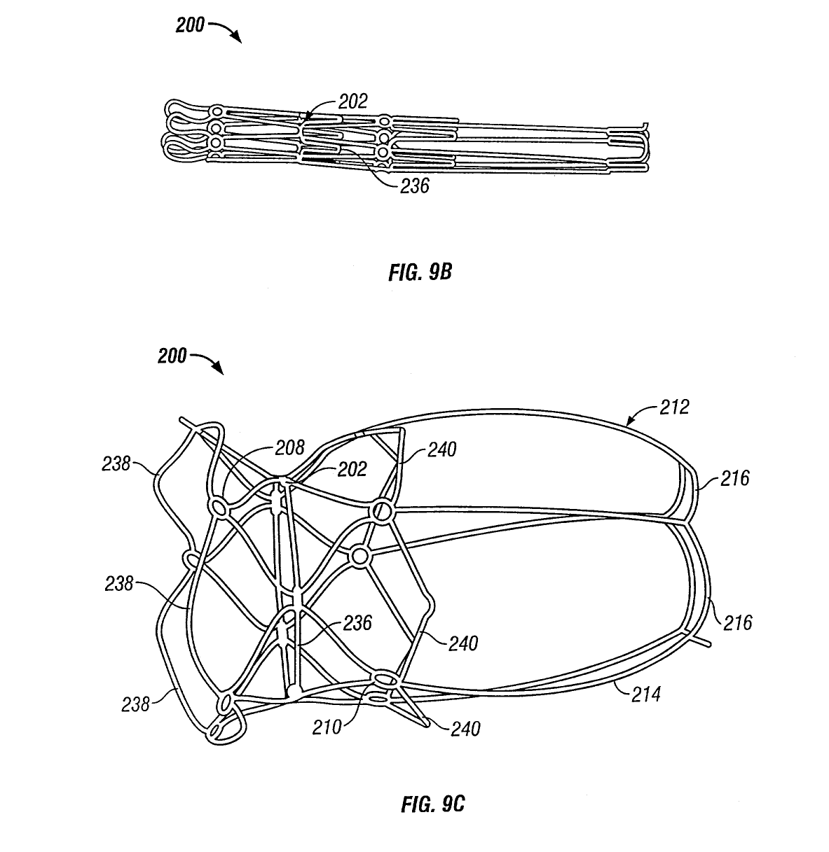

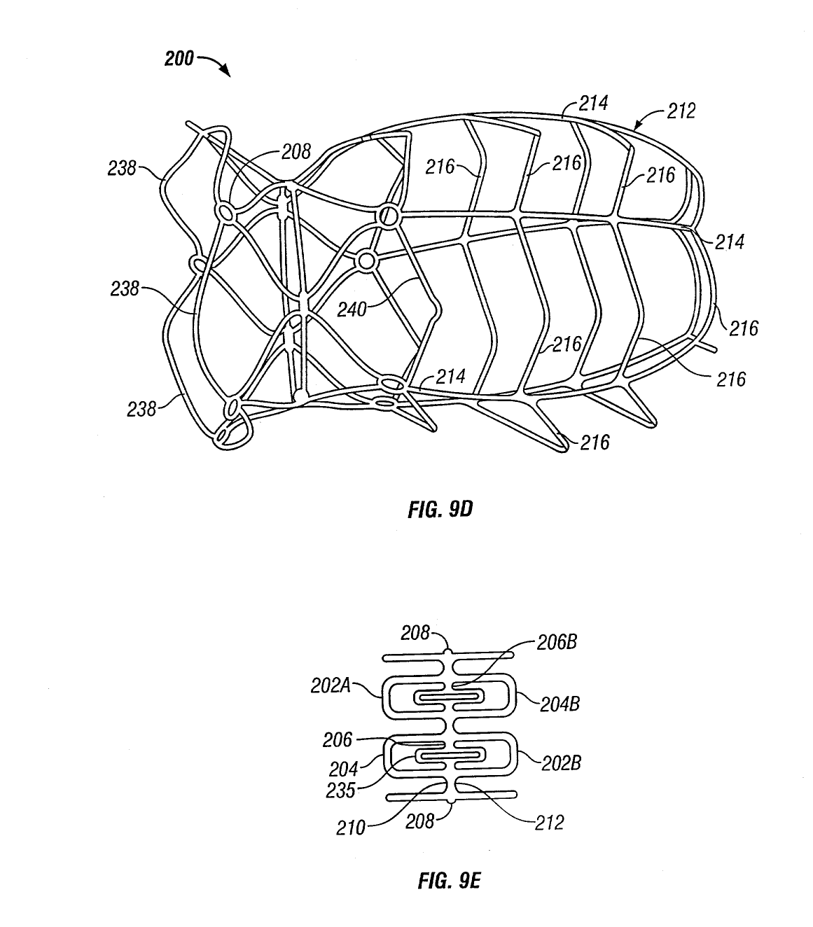

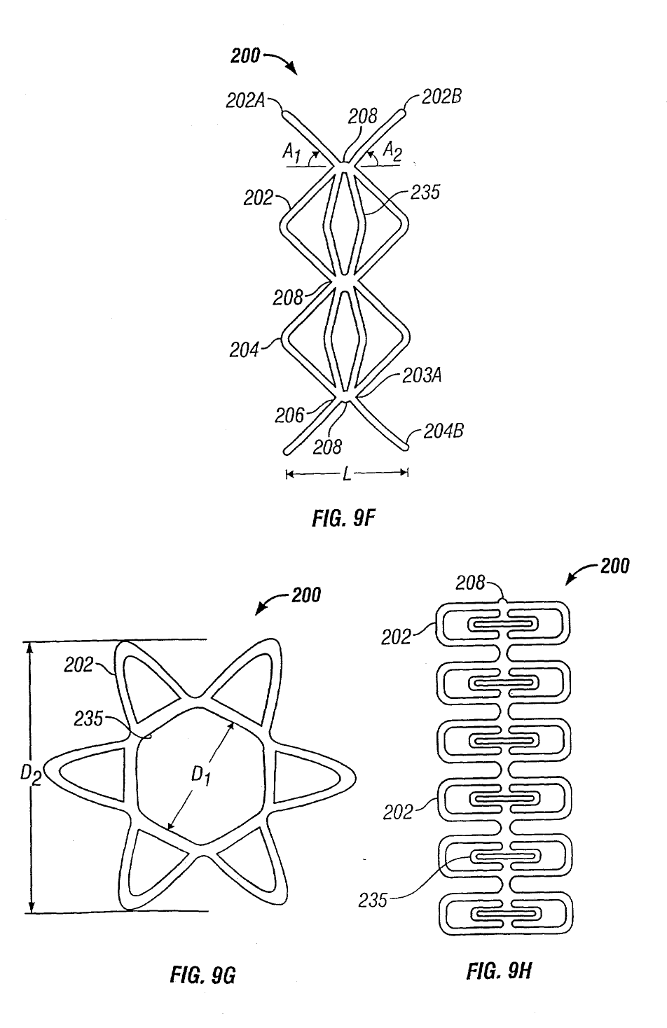

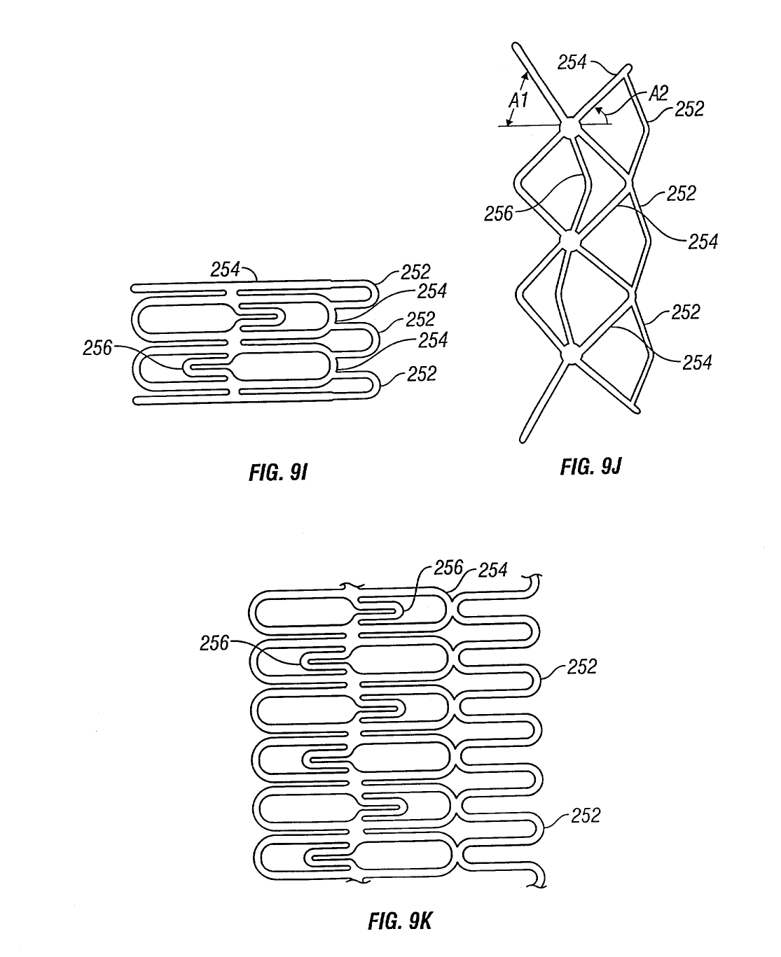

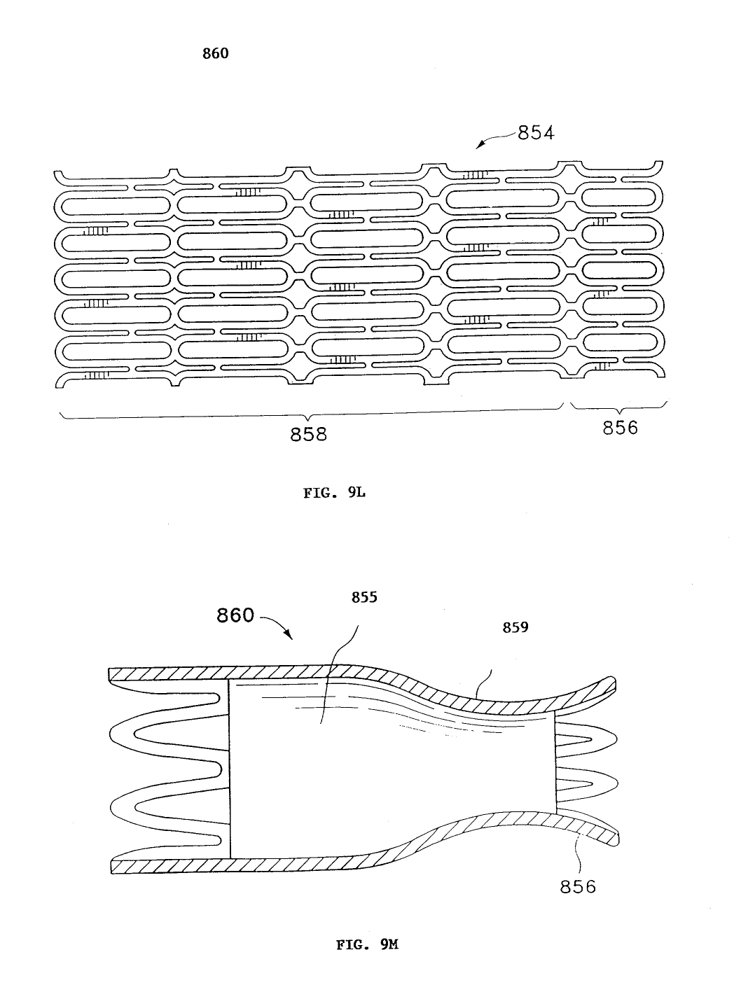

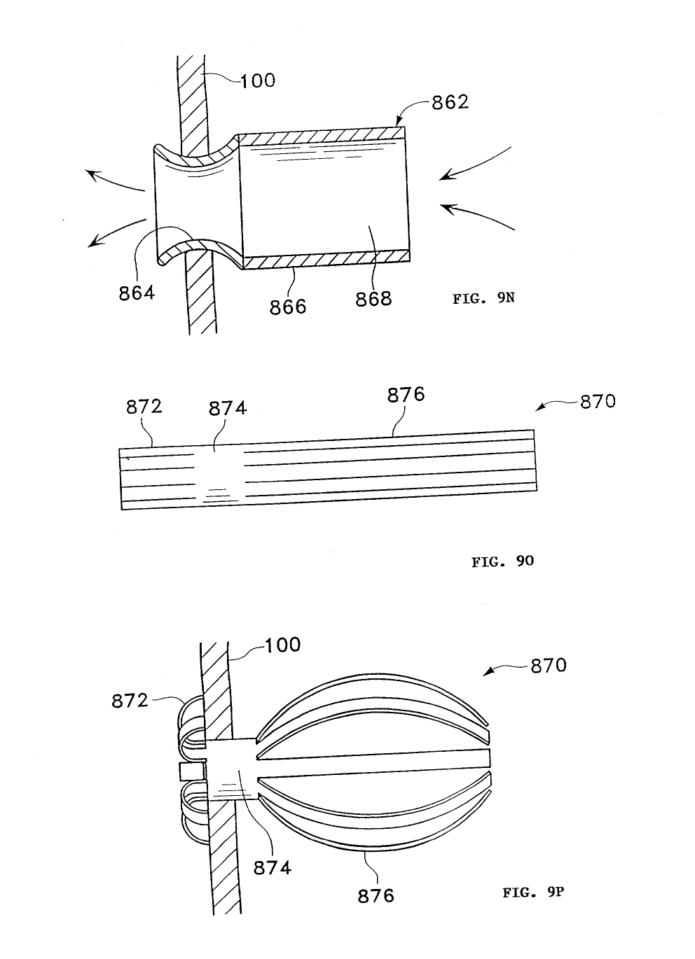

FIGS. 9A-9C illustrate an additional variation of a conduit. The conduit 200 comprises a center section 202 and proximal and distal extension members 208, 210 on either end of the center section 202. As shown in FIGS. 9A and 9B, segments 236 of the center section 202 may be folded such that a diameter of the center section 202 may be minimized prior to deployment of the conduit 200. FIG. 9C illustrates expansion of the center section 202 (usually upon deployment) where the center section segments 236 control the expansion of the diameter of the center section 202.

As noted throughout this disclosure, the number of extension members 208, 210 may vary as required. Accordingly, the number of center section segments 202 may be dependent upon the number of extension members 208, 210. However, it is contemplated that center section segments 236 may be independent from the number of extension members 208, 210. For example, a conduit 200 of the present invention may only have 1 or more such center section segments 236 within the center section 202.

FIG. 9A also illustrates proximal and distal extension members 208, 210 having proximal and distal control segments 238, 240. The control segments 238, 240 may connect at least two extension members to control expansion of the extension members upon deployment of the conduit 200. As described herein, upon deployment, the extension members 208, 210 rotate about the center section 202 thereby retaining (or sandwiching) tissue between the extension members 208, 210. The proximal and distal control segments 238, 240 may limit the extent to which the extension members 208, 210 rotate, and/or prevent them from over-expanding. Although the control segments 238, 240 are illustrated as being on the ends of the extension members 208, 210, the invention is not limited as such. For instance, the control segments 238, 240 may be placed anywhere along the extension members 208, 210.

FIG. 9A also illustrates the conduit 200 as having a cage 212 which includes a plurality of control segments 216 which interconnect at least two cage members 214 (e.g., adjacent cage members). As discussed above, the control segments 216 may control the diameter of the cage 212 during expansion and/or prevention of over-expansion of the cage 212.

It is further noted that FIGS. 9A-9C do not illustrate the inventive conduit 200 as having a tissue barrier. However, the tissue barrier is omitted for purposes of illustrating the frame of the conduit 200. It is contemplated that a tissue barrier, as described herein, may extend over the center section 202 of the conduit 200. Moreover, variations of the invention include conduits 200 with a tissue barrier located over the proximal extension members 208, the center section 202, distal extension members 210 or over any combination of these features. In such cases, the openings 242 in the extension members may be used to further anchor the tissue barrier.

FIG. 9B illustrates the conduit 200 of FIG. 9A in a pre-deployment position. FIG. 9C illustrates the conduit 200 of FIG. 9A in a deployed position. As illustrated, the proximal and distal control segments 238, 240 control expansion and rotation of the respective extension members. Also, the cage control segments 216 prevent or control expansion of the cage 212. Furthermore, FIG. 9C illustrates the expansion of center section 202 of the conduit where the center section portions 236 unfold to control expansion of the center section 202. In variations of the conduit 200 that are deployed with a balloon, the center section portions 236 assist in causing the proximal and distal extension members 208, 210 to bend about the center section to capture tissue therebetween. As discussed above, the number of control segments or center section portions is not limited to that which is illustrated. Instead, the number of these components may be varied as needed.

FIG. 9D illustrates a variation of a conduit 200 having multiple control segments 216 between adjacent cage members 214. One advantage of such a configuration is that the multiple control segments 216 may better control the expansion of the cage 212 preventing over-expansion of a portion thereof, or the control segments 216 may be configured to permit the cage 212 to assume a desired profile.

It is noted that the invention is not limited to having all of the control segments 216, 238, and 240 on each respective portion of the conduit 200. Instead, control segments 216, 238, and 240 may be found on one or more portions of the conduit 200.

Control segments may be incorporated into conduits with or without cage structures. The conduit 200 shown in FIGS. 9E-9H, for example, lacks a cage and includes diametric-control segments (tethers or leashes) 235 to control and limit the expansion of the center section 208 when deployed. This center-control segment 235 typically is shaped such that when the conduit radially expands, the center-control segment bends until it is substantially straight or no longer slack. Such a center-control segment 235 may be circular or annular shaped. However, its shape may vary widely and it may have, for example, an arcuate, semi-circular, V, or other type of shape which limits the expansion of the conduit. Also, while these figures show a center control segment on conduits without elongate cage members, the invention is not so limited. Center control segments may be incorporated into conduits having cage structures or into other types of conduits as discussed above.

Referring to FIGS. 9E-9H, one end of the center-control segment is attached or joined to the center section at one location (e.g., a first rib) and the other end of the center-control segment is connected to the center section at a second location (e.g., a rib adjacent or opposite to the first rib). However, the center-control segments may have other constructs. For example, the center-control segments may connect adjacent or non-adjacent center section members. Further, each center-control segment may connect one or more ribs together. The center-control segments may be doubled up or reinforced with ancillary control segments to provide added control over the expansion of the center section. The ancillary control segments may be different or identical to the primary control segments.

FIG. 9F illustrates the conduit 200 in a deployed configuration. As discussed above, the center-control segments 235 may bend or otherwise deform until they maximize their length (i.e., become substantially straight) such as the center-control segments 235 shown in FIG. 9F. However, the invention is not so limited and other types of center-control segments may be employed.

FIG. 9G illustrates a front view of a conduit 200 in a deployed state. The cross section of the center section is shown as a circular or hexagonal shape. However, the invention is not so limited. The cross section of an implant may be circular, oval, rectangular, elliptical, or any other multi-faceted or curved shape. Also, the inner diameter (D₁) of the center section, when deployed, may range from 1 to 10 mm and perhaps, from 2 to 5 mm. Moreover, in some variations, the cross-sectional area of the passageway, when deployed, may be between 0.2 mm² to 300 mm² and perhaps between 3 mm² and 20 mm². The diameter of the center section, when deployed, thus may be significantly larger than the passageway's axial length (e.g., a 3 mm diameter and an axial length of less than 1 mm). This ratio of the center section length to diameter (D1) may range from about 0:10 to 10:1, 0.1:6 to 2:1 and perhaps from 1:2 to 1:1.

As shown in FIGS. 9I-9K, control segments 252 may also be used to join and limit the expansion of the extension members 254 or the control segments may be placed elsewhere on the conduit to limit movement of certain features to a maximum dimension. By controlling the length of the control segments, the shape of the deployed conduit may be controlled. In the conduit shown in FIGS. 9I-9K, the conduit includes both center-control segments 256 and distal control segments 252. The center-control segments are arcuate shaped and join adjacent rib sections of the center section and the distal-control segments are arcuate and join adjacent distal extension members.

FIG. 9J illustrates the conduit in a deployed configuration and shows the various control members straightening as the extension members and center section deploy. The proximal extension members, however, are not restricted by a control member and consequently may be deflected to a greater degree than the distal extension members. Accordingly, a conduit having control members connecting, for example, regions of the center section and having additional control segments connecting extension members, may precisely limit the maximum profile of a conduit when it is deployed. This, for example, is desirable when over-expansion of the conduit is hazardous or when the deployed profile must match, conform to, or affect the tissue that the conduit is being deployed into.

This also serves to control the deployed shape of the conduit by, for instance, forcing angle A1 to differ from angle A2. Using control segments in this manner can provide for cone-shaped conduits if the various types of control-segments have different lengths. For example, providing longer proximal-control segments than distal-control segments can make angle A1 larger than angle A2. Additionally, cylindrical-shaped conduits may be provided if the center-control segments and the extension-control segments are sized similarly such that angle A1 equals angle A2. Again, the control segments straighten as the conduit expands and the conduit is thus prevented from expanding past a predetermined amount.

The angles A1, A2 may vary and may range from, for example, 30 to 150 degrees, 45 to 135 degrees and perhaps from 30 to 90 degrees. Opposing members may thus form angles A1 and A2 of less than 90 degrees when the conduit is deployed in a channel. For example, angles A1 and A2 may range from 30 to 60 degrees when the conduit is deployed.

The control segments, as with other components of the conduit, may be added or mounted to the center section or alternatively, they may be integral with the center section. That is, the control segments may be part of the conduit rather than separately joined to the conduit with adhesives or welding, for example. The control segments may also be mounted exteriorly or interiorly to the members to be linked.

FIGS. 9L-9M illustrate another variation of a conduit 860 that includes deflecting extension members 856 at only one end of the conduit 860. In this variation, the center section of the conduit may comprise a body portion 858. The conduit 860 may have a covering 855 about a portion of the conduit 860. The covering may extend throughout the length of the conduit 860 or it may be limited to a portion of the conduit 860. As illustrated in FIG. 9M, when expanded, the conduit 860 may form a reduced area 859 near the extension members 856. The conduit cross section may be designed such that a diamond pattern is formed upon expansion of the conduit 860, as illustrated in FIG. 9M.

FIG. 9N illustrates another variation of a conduit 862 having a first portion 864 and a second portion 866 and a passageway 868 extending therethrough. The first portion 864 may be a conduit design as described herein. In particular, the first portion 864 is configured to secure the conduit 862 to the airway wall 100. The first portion 864 may or may not have a center that is expandable. The walls of the first portion 864 may be fluid-tight (either through design, or a fluid tight covering) to prevent tissue in-growth through the collateral channel. Alternatively, the first portion 864 may be partially fluid-tight to facilitate tissue in-growth to improve retention of the conduit 862 to the airway wall 100. However, in the latter case, the first portion 864 should be designed to minimize tissue in-growth within the channel to prevent substantial interference with airflow through the conduit 864. As with the first portion 864, the walls of the second portion 866 of the conduit may or may not be fluid-tight. For example, the walls may be perforated or have openings through the side walls. If the second portion 866 is not fluid-tight, the larger area provides for improved airflow from lung tissue through the passageway 868 and into the airway. The second portion 866 may also be designed to be partially fluid-tight to encourage airflow through the conduit 862 but reduce the probability of blockage of the conduit 862.

The second (or cage) portion may be relatively long. For example, the cage structure may have a length ranging from 2 to 20 mm and perhaps, from 6 to 15 mm. In one configuration, the cage has an axial length of 8-12 mm and a diameter larger than that of the center section when deployed and expanded. The cage diameter in this configuration may be 1.5-4 times the diameter of the center section. Again, the conduit may be formed as an integral tubular structure or it may be formed of a plurality of ribs or mesh components which are covered with a biocompatible material to prevent tissue ingrowth.

FIGS. 9O-9P illustrate another variation of a conduit 870. The conduit 870 may be formed from a tube that is slit to form extension members at a first portion 872 and second portion 876 with a center section 874 between the portions. The conduit 870 may be expanded as shown in FIG. 9P such that the first 872 and second 876 portions maintain the center portion 874 in a collateral channel in an airway wall. The center section 874 may or may not be expandable.

FIG. 9P illustrates the second portion 876 of the conduit 870 to expand in its center. The second portion thus has a basket or cage shape. The conduit 870, however, may be designed in other configurations as well (e.g., expanded to have a larger diameter at an end opposite to the center section 874.) The second portion 870 provides a large area in the lung tissue to permit a larger volume of air to pass from the lung tissue into the conduit 870. This design has an added benefit as the second portion 876 cannot be easily blocked by flaps of parenchymal tissue. A simple variation of the conduit 870 may be constructed from a metal tube, such as 316 stainless steel, titanium, titanium alloy, Nitinol, etc. Alternatively, the conduit may be formed from a rigid or elastomeric material.

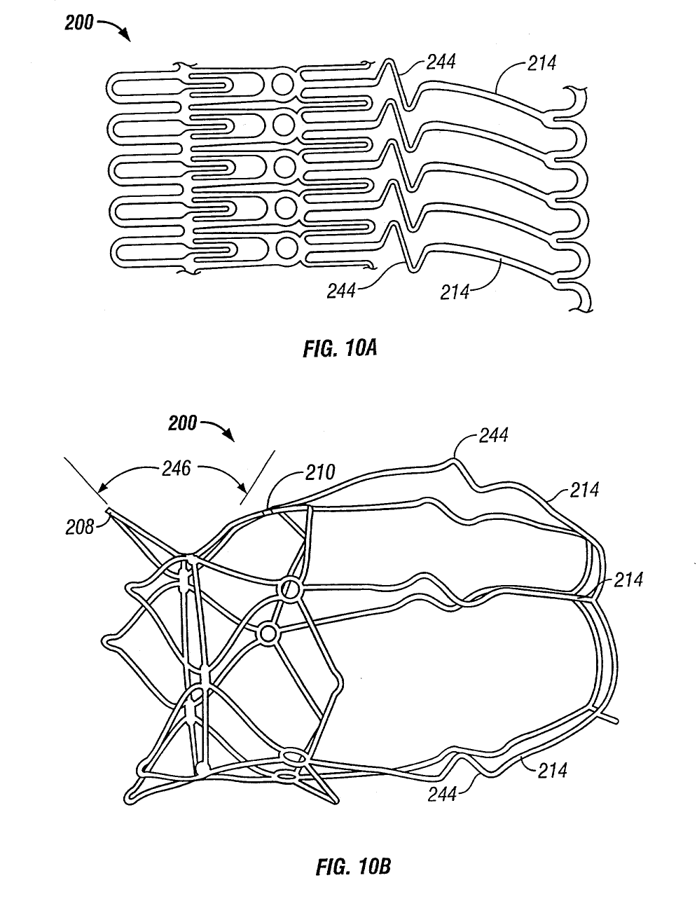

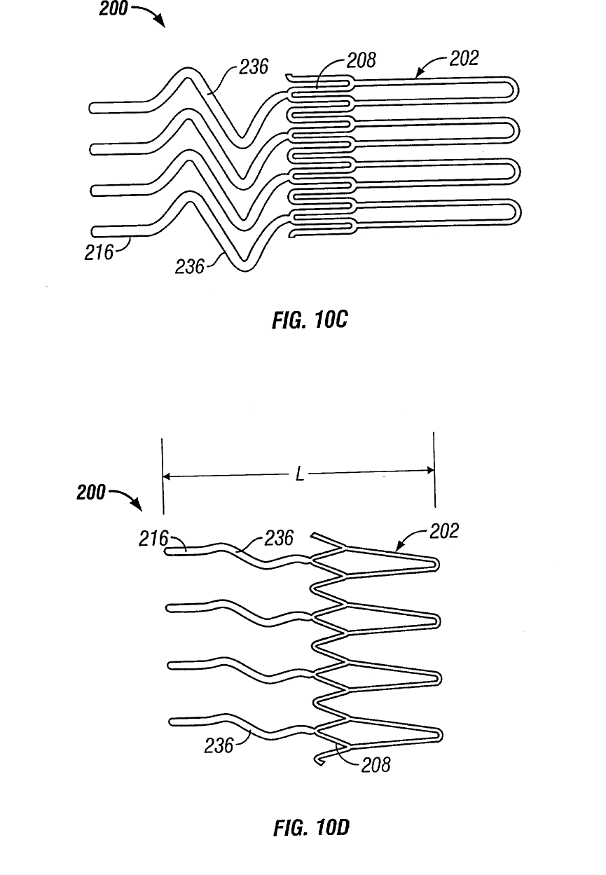

FIG. 10A illustrates another feature of the inventive conduit 200 of the present invention. As illustrated, this conduit 200 includes cage members 214 which have a length-increasing portion 244. The length-increasing portion 244 may be a section of the cage member 214 which forms a non-linear pattern. When desired, the non-linear pattern may be ‘straightened-out’ thus increasing the length of the cage member. In such cases, the non-linear pattern may eventually assume a linear pattern, or it may simply become straighter (i.e., substantially linear as compared to its original pattern or approach a linear pattern.) The length-increasing portion 244 may be formed in a sinusoidal pattern, an arc-shaped pattern, a parabolic pattern, a ‘v-shaped’ pattern, etc. It should be noted, that although the individual cage member may approach a non-linear pattern when viewed in one plane (e.g., a top view), the cage member may still have a curve when viewed in another plane (e.g., a side view) such that it forms a cage with adjacent cage members.

One benefit of the length-increasing portion 244 may be observed during the expansion of the conduit 200. In some cases, a conduit 200 may compress or shrink in an axial direction as it radially expands. This axial shrinking may resist the expansion of the conduit 200 thereby causing a larger angle for retaining tissue between corresponding proximal 208 and distal 210 extension members. If this angle is too large the conduit 200 may not be securely placed. Also, in some cases, the axial shrinking may be a benefit because it may help to sandwich tissue between the proximal and distal extension members, further securing the conduit in place. The axial shrinking may occur when elongated ribs running axially bend to accommodate the conduit's radial expansion. As the ribs bend in one direction, the conduit tends to shrink in the axial direction unless, for example, a length increasing portion or other mechanism compensates for the axial shrinking.

As shown in FIG. 10B, the use of length-increasing portions 244 permits the cage member 214 to increase in length as the cage expands, thereby allowing a smaller angle 246 to be formed between corresponding proximal 208 and distal 210 extension members. Moreover, the use of length-increasing portions 244 permits the overall conduit 200 to have a smaller axial length prior to deployment of the conduit 200 thus improving maneuverability of the conduit 200 during placement. It is not necessary for the length-increasing portion 244 to be found on each cage member 214, the invention also contemplates placement of the length-increasing portion 244 on less than all cage members 214.

FIGS. 10C-10D illustrate another conduit 200 having cage members 216 each of which has a length-increasing portion 236. The length-increasing portion 236 may be a section of the cage member 216 which forms a non-linear pattern. Unlike the cage members 214 discussed in FIGS. 10A-10B, the cage members 216 of the conduit in FIGS. 10C-10D do not include control segments to limit the expansion.

Any variation of a conduit described herein may comprise a barrier layer which is impermeable to tissue. This aspect of the invention prevents tissue in-growth from occluding the collateral channel or passage of the conduit. The barrier layer may extend between the ends of the body or the barrier layer may extend over a single portion or discrete portions of the body of the conduit.

FIG. 11 illustrates a cross section of an un-deployed conduit 200. As discussed above, it may be desirable to prevent growth of tissue through, at least, the center section 202 of the conduit 200 once it is deployed. Accordingly, an inner covering 232 may be placed within the conduit 200 and adjacent to the center section 202. The inner covering may be a tube (e.g., silicone, or other material) or a coating. In variations of the invention, the inner covering 232 may extend beyond the center section 202 and adjacent to the extension members 208, 210, or even into the cage 212. FIG. 11 also illustrates an outer covering 234 placed over the center section 202 of the conduit 200. The outer covering 234 may be a tube, coating, etc. It may be comprised of silicone or other material. Furthermore, the invention is not limited to placing the outer covering 234 only on the center section 202 of the conduit 200.

Another variation of the invention is illustrated in FIGS. 12A-12B. In this variation, a conduit contains a filler material between the openings of the ribs or mesh. For example, FIG. 12A illustrates a partial plane view of a conduit 880 having a plurality of ribs or a mesh structure 882 as previously described. The conduit 880 includes placing a filler material 884 between each of the ribs/opening of the mesh. A covering 886 is then placed over the ribs/mesh 882 and filler material 884. The covering 886 encapsulates the structure of the conduit 880 and covers the outer surface of the conduit 880 and the interior wall of the lumen or passageway of the conduit 880. FIG. 12B illustrates a partial sectional view of the conduit 880 of FIG. 12A. FIG. 12B illustrates the mesh 882 with filler material 884 adjacent to the mesh 882 and an outer covering 886 encapsulating the mesh 882 and filler material 884. It is noted that the filler material 884 and covering 886 may be placed entirely throughout a conduit. Alternatively, the filler material 884 and covering 886 may be placed partially over a conduit as needed. It is believed that the addition of filler material to a conduit provides a uniform thickness of the covering which results in uniform and consistent stretching of the covering. Some various examples of filler material are, for example, wax, silicone, and urethane. The covering may consist of, for example, silicone, urethane, or similar materials.

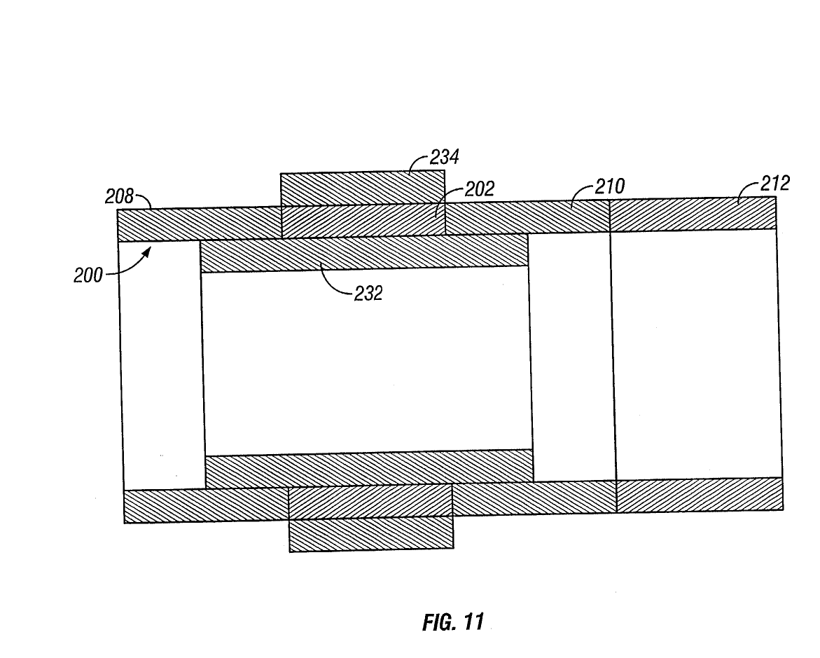

FIGS. 13A-13C illustrate another variation of a conduit 888. As shown in FIG. 13A, the wall thickness of the material 896 between the extension members 890 and the center section 892 may be less than a thickness of the wall section of the material 896 at the center section 892. As illustrated, if an outwardly radial force is applied to the extension members 890, such a configuration results in bending at the area of reduced wall thickness 894. Consequently, and as illustrated in FIGS. 13B-13C, the extension members 890 expand in a predetermined manner. The material may be elastomeric and perhaps, urethane or silicone. Reduced thickness areas may also be used in metal frame conduits to encourage bending at pre-specified locations.

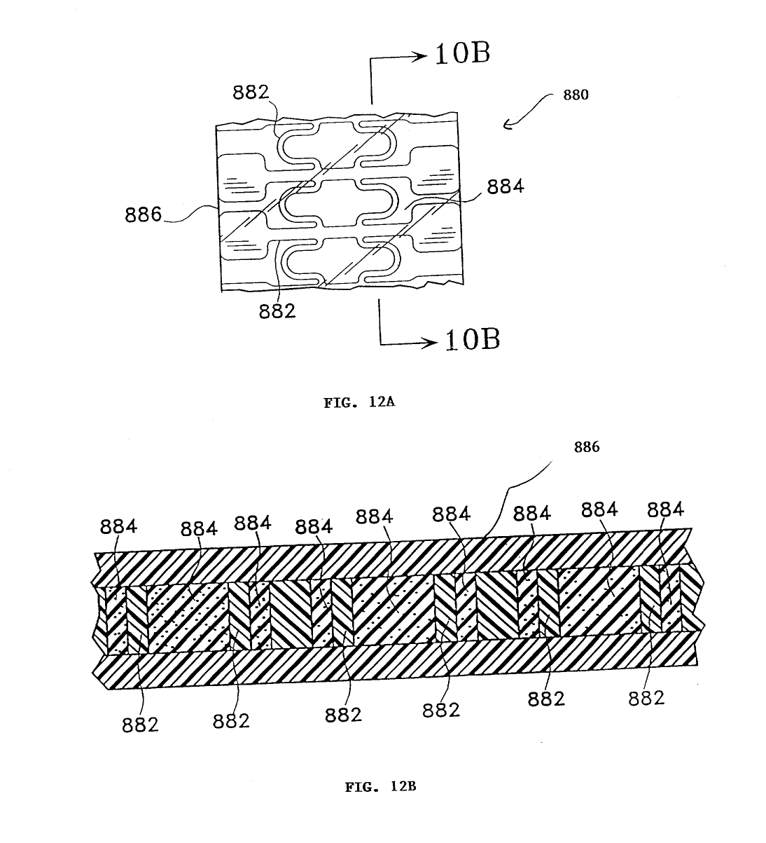

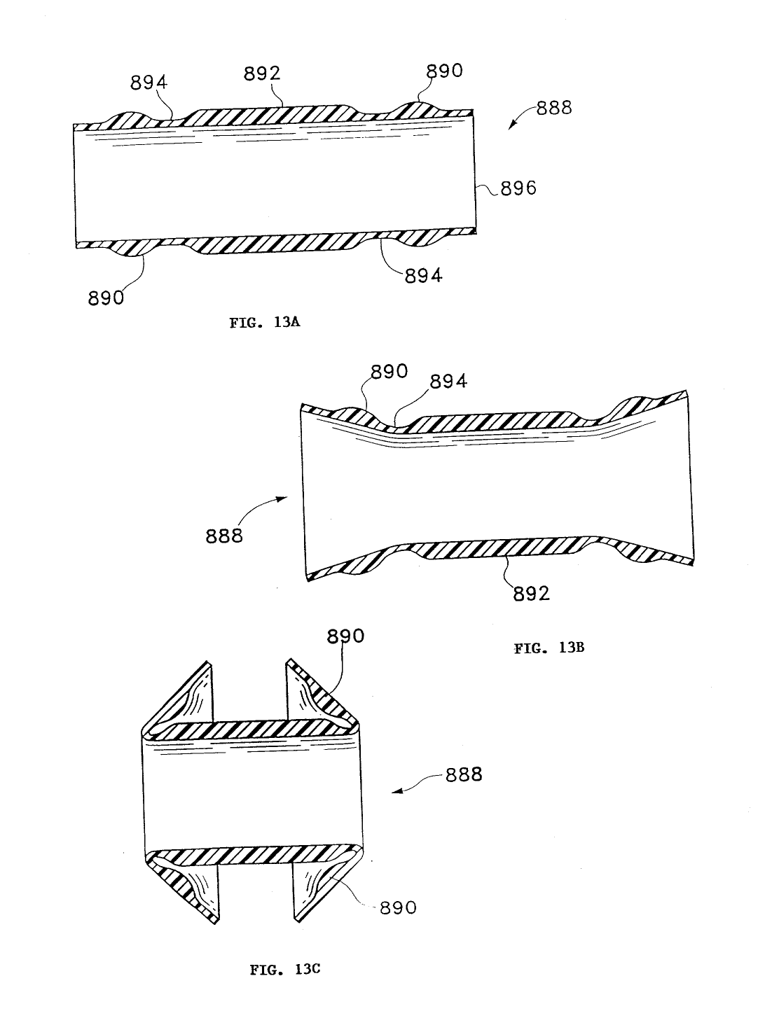

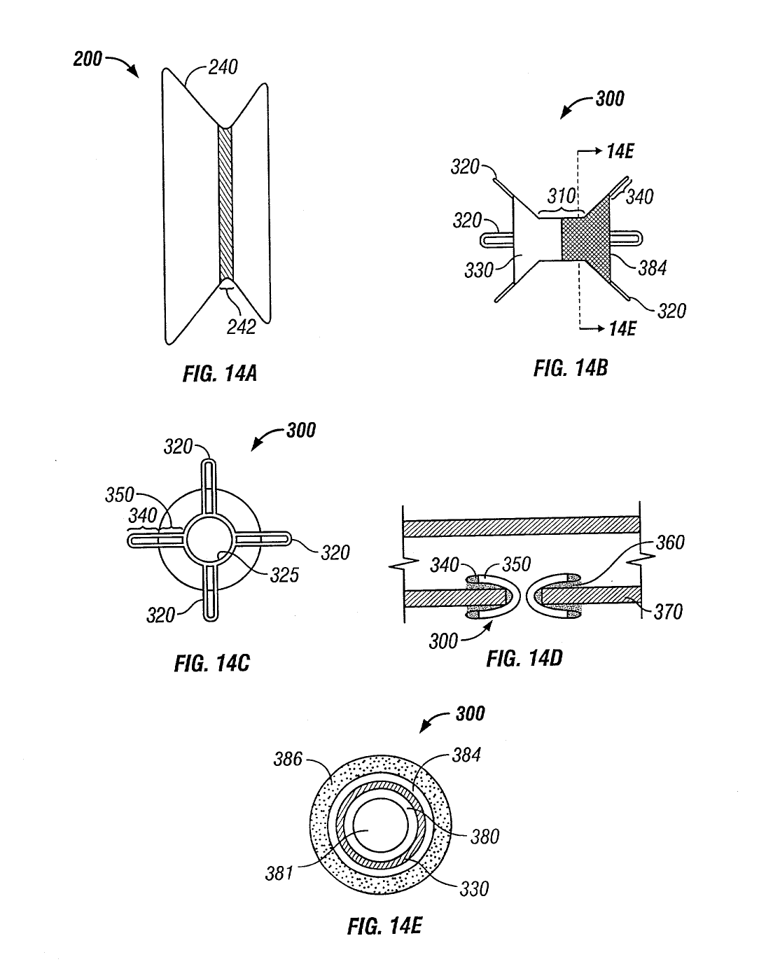

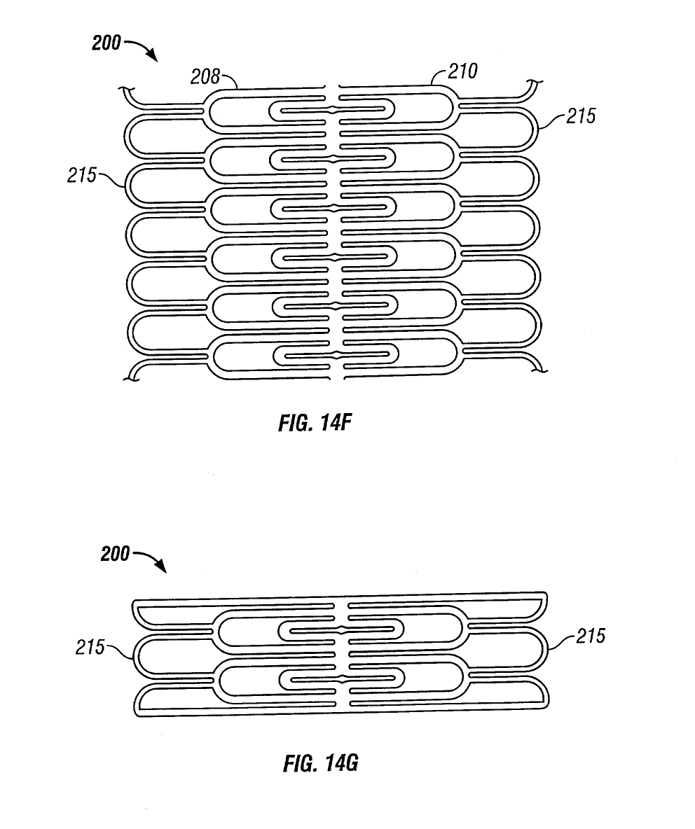

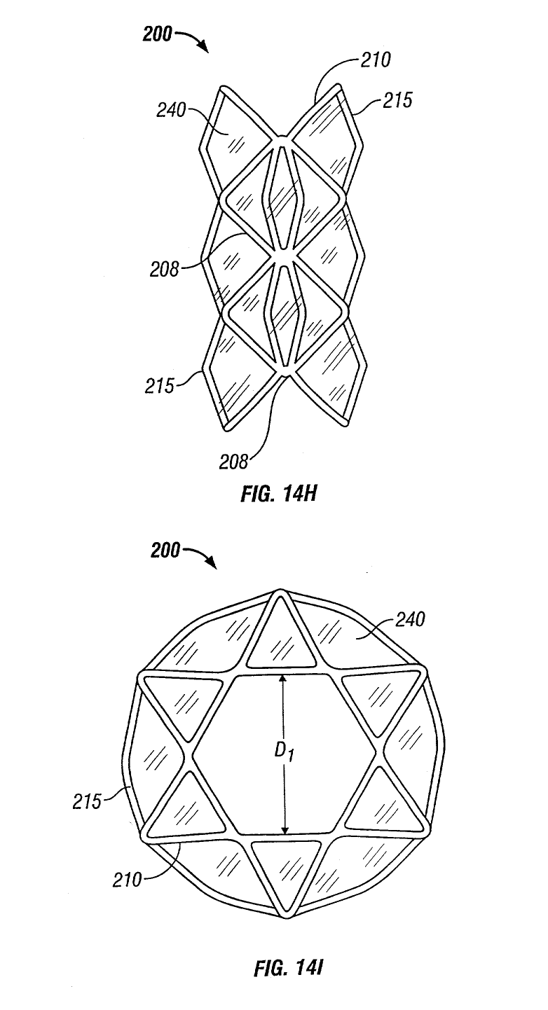

FIG. 14A illustrates another variation of a conduit 200 having a tissue barrier 240. The tissue barrier 240 prevents tissue ingrowth from occluding the collateral channel or passage of the conduit 200. The tissue barrier 240 may coaxially cover the center section from one end to the other or it may only cover one or more regions of the conduit 200. Thus, the tissue barrier may completely or partially cover the conduit. The tissue barrier 240 may be located about an exterior of the conduit's surface, about an interior of the conduit's surface, or the tissue barrier 240 may be located within openings in the wall of the conduit's surface as described above. Furthermore, in some variations of the invention, the center section 208 itself may provide an effective barrier to tissue ingrowth. The tissue barrier, of course, should not cover or block the entrance and exit of the passageway such that air is prevented from passing through the conduit's passageway. However, in some constructs, the tissue barrier may partially block the entrance or exit of the passageway so long as air may continue to pass through the conduit's passageway.

The tissue barrier may be formed from a material, or coating that is a polymer or an elastomer such as, for example, silicone, polyurethane, PET, PTFE, or expanded PTFE. Moreover, other biocompatible materials may be used, such as a thin foil of metal, alloy, etc. The coatings may be applied, for example, by either dip coating, molding, spin-coating, transfer molding, compression molding or liquid injection molding. Or, the tissue barrier may be a tube of a material and the tube is placed either over and/or within the conduit. The tissue barrier may then be bonded, crimped, heated, melted, fused or shrink fitted to the conduit. The tissue barrier may be tied to the conduit with a filament of, for example, a suture material. The tissue barrier may be placed on the conduit by either solvent swelling applications or by an extrusion process. Also, a tissue barrier may be applied by either wrapping a sheet of material about the conduit, or by placing a tube of the material about the conduit and securing the tube to the conduit. Likewise, a tissue barrier may be secured on the interior of the conduit by positioning a sheet or tube of material on the inside of the center section and securing the material therein.

FIGS. 14B and 14C respectively illustrate a side view and a front view of another conduit 300 having a partial tissue barrier coating. The conduit 300 includes a center section 310, a plurality of extension members 320, and a partial tissue barrier 330. The conduit 300 is thus different than that shown in FIG. 14A in that the center section is longer and that the tissue barrier 330 only partially covers the extension members 320. The center section 310 shown in FIGS. 14B-14C is cylindrical or tubular-shaped. This shape may be advantageous when a relatively longer passageway is desired. Also, it is to be understood that the overall (or three dimensional) shape of the center section, when deployed, is not limited to the shape shown here. Rather, it may have various shapes such as, for example, rectangular, tubular, conical, hour-glass, hemi-toroidal, etc. Also, cage members may be connected to the center section or to the extension members. The barrier may cover the cage members as well. The cage members may be coaxially covered partially or completely by a tissue barrier.

Referring to FIGS. 14B-14C the tissue barrier 330 covers only a central region 350 of the extension members and leaves a free or end region 340 of the extension members uncovered. The end region 340 of the extension members 320 is shown as being open-framed. However, the invention is not so limited. The distal region of the extension members may be solid and it may include indentations, grooves, and recesses for tissue ingrowth. Also, the extension members may include small holes for tissue ingrowth. For example, the distal region of the extension members may have a dense array of small holes. In any event, the conduits described herein may include at least one region or surface which is susceptible to tissue ingrowth or is otherwise adherent to the tissue. Accordingly, tissue ingrowth at the distal region 340 of the extension members is facilitated while tissue growth into the passageway 325 is thwarted.

As shown in FIG. 14D, tissue growth 360 into the uncovered region 340 further secures the extension members to the tissue wall 370. The distal region of the extension members may also include tissue growth substances such as epithelial growth factors or agents to encourage tissue ingrowth. Accordingly, conduit 300 may be configured to engage the tissue wall 370 as well as to allow tissue to grow into predetermined regions of the conduit.

The conduit shown in FIG. 14A also includes a visualization ring or marker 242. The marker 242 is visually apparent during a procedure. The marker is observed as the conduit is placed in a collateral channel and, when the marker is even with the opening of the channel, the conduit may be deployed. In this manner, the visualization feature facilitates alignment and deployment of the conduits into collateral channels.

The visualization ring or mark may be a biocompatible polymer and have a color such as white. Also, the visualization feature may protrude from the center section or it may be an indentation(s). The visualization mark may also be a ring, groove or any other physical feature on the conduit. The visualization feature may be continuous or comprise discrete segments (e.g., dots or line segments).

The visualization feature may be made using a number of techniques. In one example, the mark is a ring formed of silicone and is white. The polymeric ring may be spun onto the tissue barrier. For example, a clear silicone barrier may be coated onto the conduit such that it coaxially covers the extension members and the center section as shown in FIG. 14A. Next, a thin ring of white material such as a metal oxide suspended in clear silicone may be spun onto the silicone coating. Finally, another coating of clear silicone may be applied to coat the white layer. The conduit thus may include upwards of 1-3 layers including a tissue barrier, a visualization mark layer, and a clear outer covering.

The shape of the visualization mark is not limited to a thin ring. The visualization mark may be large, for example, and cover an entire half of the conduit as shown in FIG. 14B (see reference numeral 384 of FIG. 14B). The visualization mark may, for example, be a white, black, blue or another opaque coating disposed on the proximal or distal half of the conduit. The visualization mark thus may extend from an end of the extension members to the center section of the conduit. As explained in more detail below, when such a device is deposited into a channel created in lung tissue, the physician may observe when one-half of the conduit extends into the channel. This allows the physician to properly actuate or deploy the conduit to secure the conduit in the tissue wall.

The visualization feature is made visually apparent for use with, for example, an endoscope. The visualization feature, however, may also be made of other vision-enhancing materials such as radio-opaque metals used in x-ray detection. It is also contemplated that other elements of the conduit can include visualization features such as but not limited to the extension members, cage members, tissue barrier, control segments, etc.

The conduits described herein may include modified surfaces that prevent the channel from closing by reducing tissue growth into the passageway. The modified surfaces may also prevent the conduit from being ejected from the channel as the wound heals. The surfaces of the conduit may be modified, for example, by depositing a bioactive substance or medicine onto the exterior surface of the conduit.

The bioactive substances are intended to interact with the tissue of the surgically created channels. These substances may interact with the tissue in a number of ways. They may, for example, accelerate wound healing such that the tissue grows around the exterior surface of the conduit and then stops growing; encourage growth of the epithelial or endothelial cells; inhibit wound healing such that the injury site (e.g., the channel or opening) does not heal leaving the injury site open; and/or inhibit infection (e.g., reduce bacteria) such that excessive wound healing does not occur which may lead to excessive tissue growth at the channel thereby blocking the passageway. However, the foregoing statements are not intended to limit the present invention and there may be other explanations why certain bioactive substances have various therapeutic uses in the lung tissue. Again, the bioactive substances are intended to prevent the implant from being ejected as well as prevent the lung tissue from filling or otherwise blocking the passageway of the conduit.

A variety of bioactive substances may be used with the devices described herein. Examples of bioactive substances include, but are not limited to, pyrolitic carbon, titanium-nitride-oxide, paclitaxel, fibrinogen, collagen, thrombin, phosphorylcholine, heparin, rapamycin, radioactive 188Re and 32P, silver nitrate, dactinomycin, sirolimus, cell adhesion peptide. Again, other substances may be used with the conduits such as those substances which affect the wound healing response (or rate) of injured tissue or which affect any physiological, biological, or mechanical characteristic of tissue such as tissue modulus or elasticity, cell regeneration rate, smooth muscle contractibility, etc.

A cross section of a conduit 300 having a modified surface is shown in FIG. 14E. In particular, the conduit 300 comprises an inner frame layer or ribs 380 which define a passageway 381 for air to flow through. Coaxially surrounding the frame 380 is a tissue barrier 330. Additionally a visualization coating 384 is disposed on a portion of the tissue barrier 330. The visualization coating 384 is deposited as described above. A bioactive substance 386 may be deposited on one or both the visualization and tissue barrier layers either directly or via a binding layer as described below. In this manner, the bioactive substance is disposed on an exterior surface of the conduit and contacts tissue when the device is deployed in a channel. However, it is contemplated that additional layers may be added such as, for example, an additional silicone layer over the visualization layer. Typically, the bioactive layer will form the outer-most layer.

Also the order of the layers may be different than that described above. For example, the visualization layer may be disposed over the bioactive layer. Also, not all coatings and materials shown in FIG. 14E are necessary to carry out the present invention. For instance, the bioactive substances in some cases may be deposited directly on the open-frame 380.

The bioactive layer may also serve as the visualization coating or tissue barrier in some instances. For example, silicone and one or more bioactive substances may be mixed together and disposed on the conduit as a single coating. The single integral layer may serve both to physically and chemically prevent tissue from filling the conduit's passageway. It may also be visually apparent during a procedure.

Additionally, the bioactive substances may be deposited on the exterior surface of the conduit evenly or in discrete (intermittent) amounts. The thickness of the coatings may be uniform or the thickness may vary across certain regions of the conduit. This may provide higher therapeutic doses corresponding to certain regions of the injury site. For example, it may be desirable to provide a higher concentration of a bioactive substance near the ends of the conduit rather than in the center section.

The bioactive coatings may be selectively applied by spraying the bioactive substance onto uncovered regions of the conduit. For example, the bioactive substances may be disposed on at least a portion of the tissue barrier or the open-frame (or mesh) structure itself. The substances may also be applied by dipping, painting, printing, and any other method for depositing a substance onto the conduit surface. Additionally, binding materials may be applied to the exterior surface of the conduit upon which the bioactive agents may be deposited. Cross-linked polymers and or biodegradable polymers such as, for example, chondroitin sulfate, collagen and gelatin may be applied to the exterior surface of the conduit prior to depositing the bioactive substances. Additionally, the exterior surface of the conduit may be treated via etching processes or with electrical charge to encourage binding of the bioactive substances to the conduit.

Again, the bioactive substances may be deposited on the exterior of the conduits to prevent ejection of the conduit from the injury site. The bioactive substances serve to reduce or impede tissue growth into the conduit's passageway. In this manner, the conduits maintain the patency of channels surgically created in the lung airways allowing air to pass therethrough.

FIGS. 14F-14I illustrate a variation of a conduit 200 having membrane-supports 215. The membrane-supports serve to prevent the tissue barrier from tearing between the extension members 208, 210.

FIGS. 14F and 14G show the conduit 200 without a tissue barrier to better illustrate the frame of the conduit. In particular, FIG. 14F shows a planar projection of a conduit 200 in an undeployed state. FIG. 14G shows a side view of the conduit 200 in an undeployed state.

FIGS. 14H and 14I illustrate the conduit 200 in a deployed state. In particular, FIG. 14H is a side view of the conduit 200 in a deployed state and FIG. 14I shows a front view of the conduit shown in FIG. 14H. As shown, membrane-supports 215 straighten when the extension members 208, 210 are rotated into a deployed configuration. The membrane-supports 215 support the tissue barrier 240 between the extension members as the tissue barrier is stretched between the extension members during deployment. Accordingly, the membrane-supports serve to prevent tearing of the tissue barrier during deployment of the conduit 200. These membrane-supports may be incorporated into other designs described herein. The membrane-supports are connected or otherwise formed between the extension members or cage members. More particularly, the membrane-supports may be positioned at or near the ends of the coating or tissue barrier. The membrane-supports may also extend from the ends of the extension members or cage members and connect adjacent members together. In this manner, and when the conduit is coated with a tissue barrier, the membrane-supports form a bridge across deployed extension members and cage members preventing tearing of the tissue barrier.

In contrast to diameter-control members described above, membrane-support members may not act to control the shape of the conduit and may remain substantially curved or non-straight upon deployment. Again, the membrane-support members can provide a bridge across the deployed extension members preventing tearing of the tissue barrier.

FIGS. 14J and 14K illustrate another conduit 200 having features which serve to prevent tearing of the tissue barrier during deployment of the conduit. In particular, the conduit 200 includes tapered extension members 208, 210 which, because of their reduced profile as compared to untapered extension members, allow more (and perhaps thicker) tissue barrier material to occupy the space between the extension members. When the extension members are deployed as shown in FIG. 14K, the tissue barrier 240 stretches from one extension member to the next. The tissue barrier may be less likely to tear between the deployed extension members due to the increased amount of tissue barrier material between the extension members as compared to conduits which have full size, untapered extension members. Tapered extension members or tapered petals may be incorporated into other conduit designs described herein. Also, cage members may have tapered ends to reduce tearing between the cage members when the conduit is deployed.

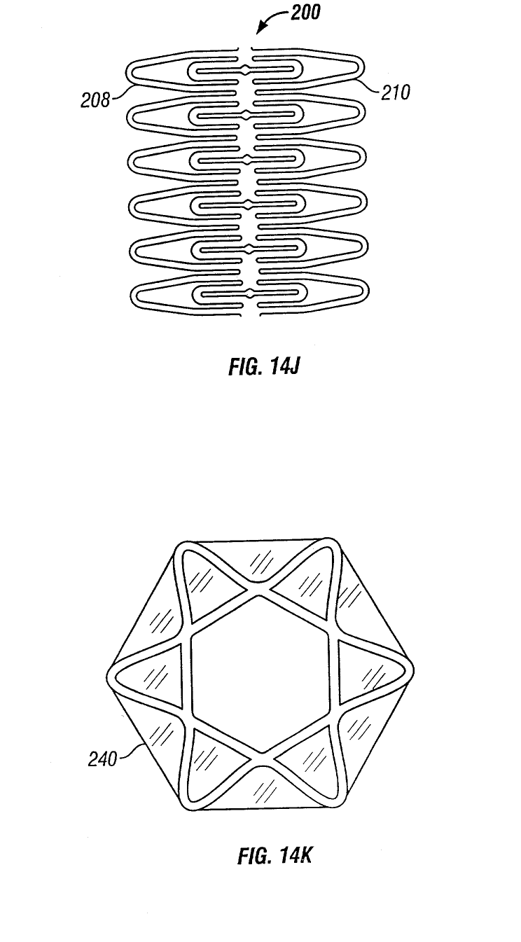

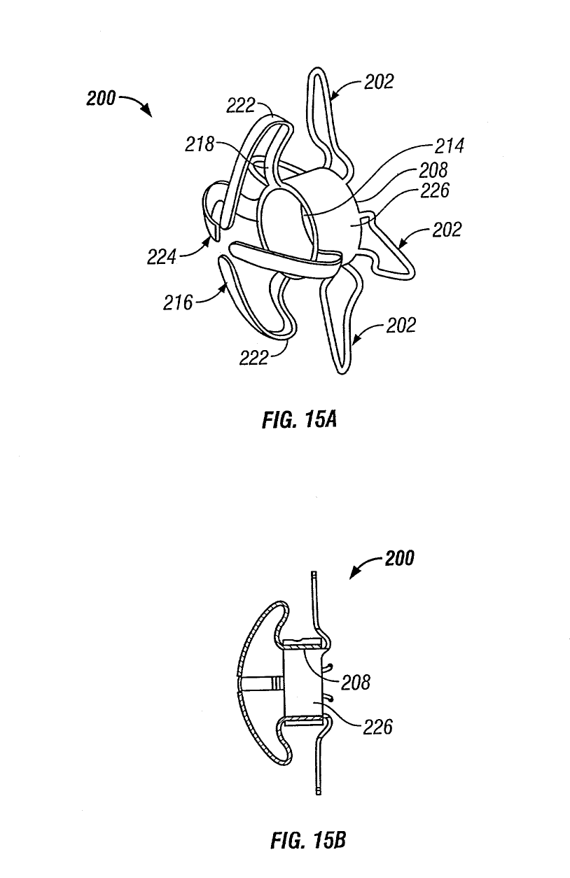

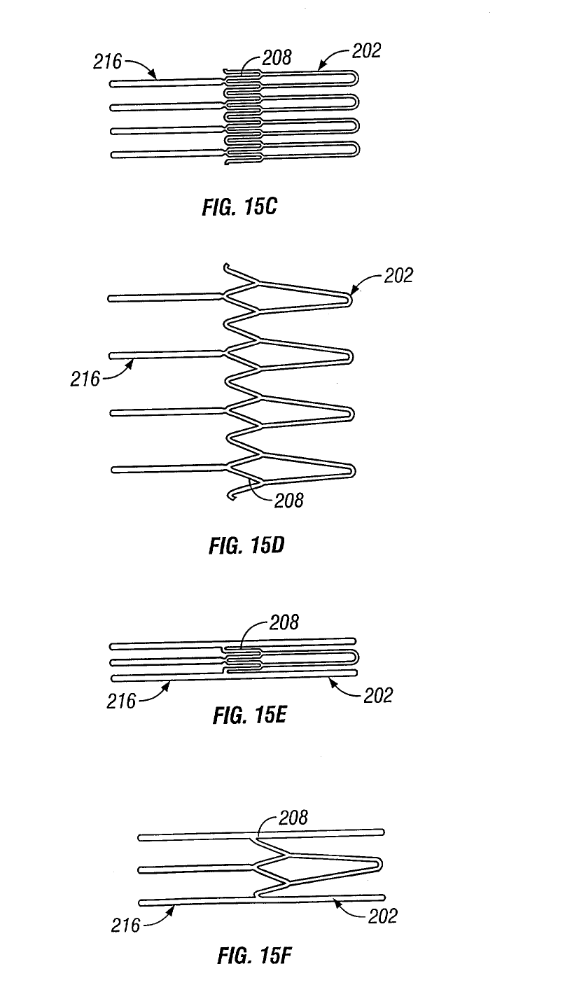

FIGS. 15A-15F illustrate another variation of a conduit 200. FIG. 15A provides a perspective view of a conduit 200 after deployment. The conduit 200 includes proximal extension members 202 and cage members 216 on either side of a center section 208. In this variation, a portion of the cage members 216 located between the fixed end 218 and the intermediate segment 222 is adapted to assume a position substantially transverse to the passage. In such a case, the portion serves a similar function as the proximal extension members 202 in that tissue is retained between the cage members 216 and the proximal extension members 202 thereby retaining the conduit 200 within an airway wall. As illustrated in the figure, the remainder of the cage member 216 may form a cage structure 224.

FIG. 15A also illustrates the inventive conduit 200 as having a tissue barrier 226 located about the center section 208 of the conduit 200. Variations of the inventive conduit may include such a tissue barrier 226 to prevent tissue in-growth from occluding the collateral channel or passage of the conduit 200. The tissue barrier 226 may extend between the ends of the center section 208 or it may cover various portions of the conduit 200.

FIG. 15B illustrates a cross-sectional view of the conduit 200 of FIG. 15A. As illustrated, the tissue barrier 226 is located about an exterior of the center section 208 of the conduit 200. However, the invention is not limited as such, for example the tissue barrier 226 may be located about an interior of the center section, or the barrier 226 may be located within an opening of the center section. Furthermore, in some variations of the invention, the center section 208 itself may provide an effective barrier to tissue in-growth.

FIGS. 15C and 15D illustrate a cylindrical projection view of the conduit 200 of FIG. 15A without a tissue barrier. These planar views show the conduit 200 in a flat and “un-rolled” state for purposes of illustration. FIG. 15C illustrates the conduit 200 prior to deployment. The members comprising the center section 208 are in close proximity, thus allowing the center section 208 to assume a smaller profile during, for example, delivery to an implant site. FIG. 15D illustrates the conduit 200 after expansion of the center section 208 and proximal members 202.

FIGS. 15E and 15F illustrate a side view of the conduit 200 of FIG. 15A prior to deployment of the proximal extension members 202 and cage members 216. For purposes of illustration, the conduit 200 is shown without a tissue barrier. FIG. 15E shows the center section 208 in a reduced profile state. FIG. 15F shows the center section 208 in an expanded profile state (as compared to FIG. 15E). FIG. 15F also illustrates a variation of the invention where the proximal extension members 202 expand as the center section 208 expands.

Accordingly, the conduits 200 of the present invention may have center sections that are expanded into a larger profile from a reduced profile, or, the center sections may be restrained in a reduced profile, and upon release of the restraint, return to an expanded profile. The conduits of the present invention may be comprised from, for example, a shape-memory alloy, a super-elastic alloy, stainless steel, titanium, titanium alloy, nitinol, MP35N (a nickel-cobalt-chromium-molybdenum alloy), polymers, any implantable material etc.

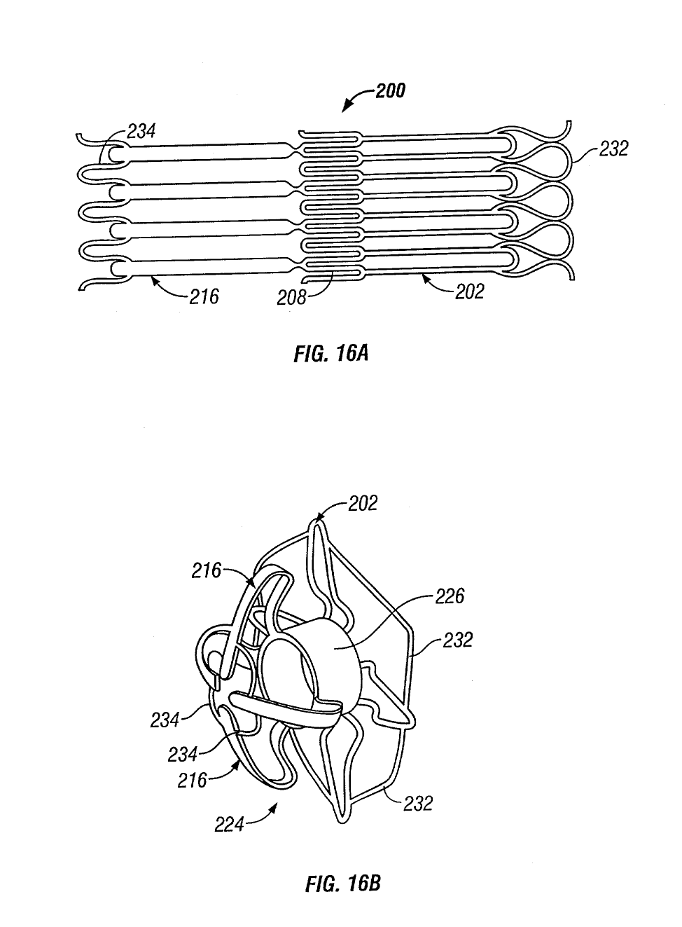

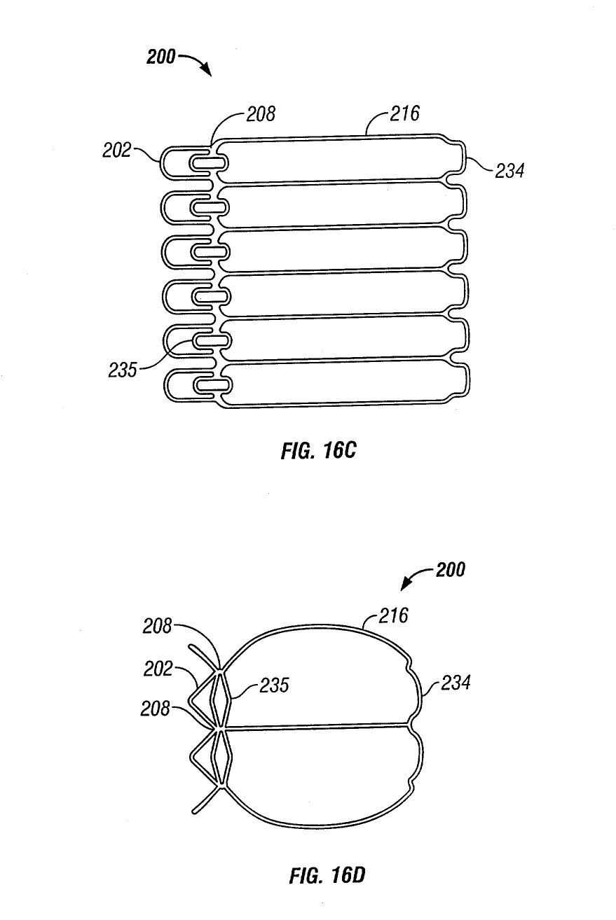

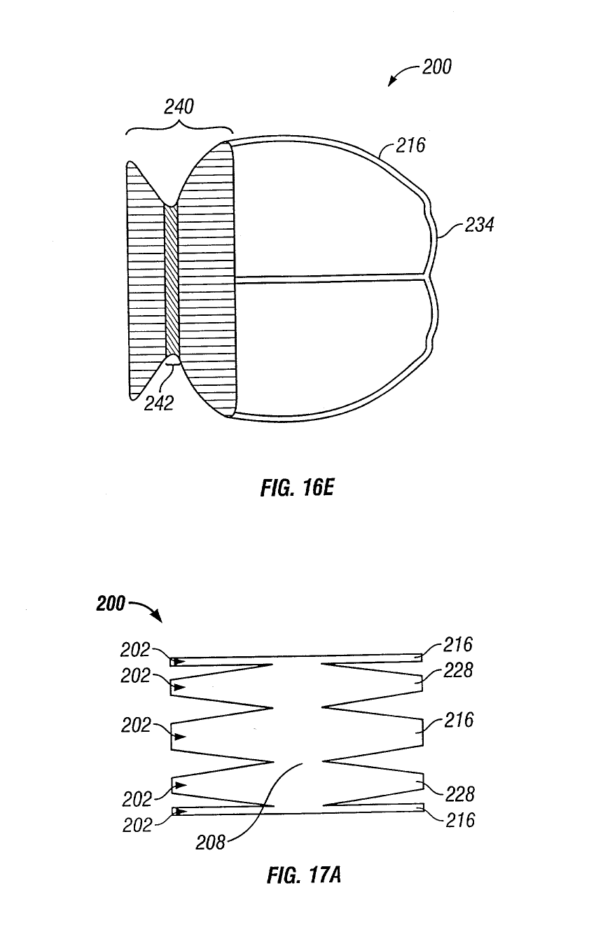

FIG. 16A illustrates a planar view of another variation of a conduit 200. The conduit 200 comprises a center section 208 having proximal extension members 202 and cage members 216 on either end of the center section 208. As noted throughout this disclosure, the number of extension members 202, 216 may vary as required. In this illustration, the movable or free ends of both the proximal extension members 202 and the cage members 216 include proximal control segments 232 and cage control segments 234 respectively. The control segments 232, 234 may connect at least two extension members to control expansion and/or formation of the extension members upon deployment of the conduit 200. As described herein, upon deployment, the proximal extension members 202 and cage members 216 may rotate about the center section 202. The proximal control segments 232 and cage control segments 234 may limit the extent to which the extension members 202, 216 rotate, or prevent them from over-expanding, etc. The control segments 232, 234 may also assist the conduit 200 in assuming a desired shape and for maintaining structural rigidity of the conduit 200 when implanted. Although the control segments 232, 234 are illustrated as being on the ends of the extension members 202, 216, the invention is not limited as such. For instance, the control segments 232, 234 may be placed anywhere along the extension members 202, 216. The control segments may also be placed at the center section to limit the expansion of the center section as described above.

FIG. 16B illustrates the control segments 234 located on the cage members 216 assisting in formation of the cage 224. The control segments 234 may control the diameter of a portion of the cage 224 during expansion and/or prevention of over-expansion of the portion of the cage 224. In the variation of the invention depicted in FIG. 16B, the control segments 234 cause an end of the cage structure 224 to form a smaller profile than a center of the cage structure 224. It is noted that various other configurations are considered to be within the scope of this invention. FIG. 16B also illustrates how the-proximal control segments 232 control expansion and rotation of the respective extension members.

It is noted that the invention is not limited to having control segments 232, 234 on each extension member 202, 216 of the conduit 200. Instead, the control segments 232, 234 may be found on one or more extension members 202, 216 of the conduit 200. Moreover, more than one control segment may be attached to the extension members.

FIG. 16C illustrates another conduit 200 having proximal extension members 202, a center section 208, and a plurality of basket or cage members 216. The cage members are shown joined with segments 234. The conduit 200 shown in FIG. 16C also includes center-control segments 235 to control and limit the expansion of the center section when deployed. In this case, the center-control segment is arcuate or circular-shaped. However, the center-control segments may vary. For example, the center-control segment may have a semi-circular, “V”, or other-type of shape which provides for limited expansion. Further, the center-control segments may connect one or more of the center section members together. Also, the center-control segments may connect adjacent or non-adjacent center section members. The center-control segments may further be doubled up or reinforced with ancillary control segments to provide added control over the expansion of the center section. The ancillary control segments may be different or identical to the primary control segments.

FIG. 16D illustrates the conduit 200 in its deployed configuration. When deployed, the center-control segments 235 bend into another configuration. The center-control segments 235 shown in FIG. 16D are deformed to a diamond shape as each curved (or arcuate) segment straightens. However, as discussed above, the invention is not so limited and includes other configurations of center-control segments.

FIG. 16E shows another conduit having a tissue barrier 240 and a visualization ring 242. The tissue barrier 240 covers center section 208 and prevents tissue in-growth into the conduit. The tissue barrier 240 shown in FIG. 16E also covers the proximal extension members 202 and a portion of cage member 216. The tissue barrier may be a biocompatible polymer as described herein and prevents tissue and mucous from closing or blocking the passage of the conduit. While the tissue barrier is shown covering all of the proximal extension members, the invention is not so limited. The tissue barrier may cover more or less of the conduit so long as it does not prevent air from passing through the passage.

The conduit shown in FIG. 16E also includes a visualization ring or marker 242. The visualization marker may be similar to that described above. The visualization feature may be added to the conduit using various materials including, but not limited to, biocompatible inks, paints, epoxy, metals, metal oxides, alloys, polymers or combinations thereof. An ink pen, for example, may be used to draw a visualization feature onto the conduit. Also, a material may be wrapped around the conduit or embedded in a tissue barrier to form the visualization feature. Additionally, the visualization feature may be made of other detectable materials such as radio-opaque metals used in x-ray detection.

Accordingly, the visualization feature of the present invention may be added to the center section of the conduit and perhaps to the tissue barrier surrounding the center section to facilitate visualization and deployment of the conduit. It is also contemplated that other elements of the conduit can include visualization features such as but not limited to the extension members, tissue barrier, control segments, etc.

The conduit shown in FIGS. 16C-16E is fabricated, used and deployed similar to the conduits described herein. Also, aspects of the conduit 200 shown in FIGS. 16C-16E (e.g., the visualization ring or the control segments) may be used in combination with other examples set forth in this description except, of course, where features are mutually exclusive.

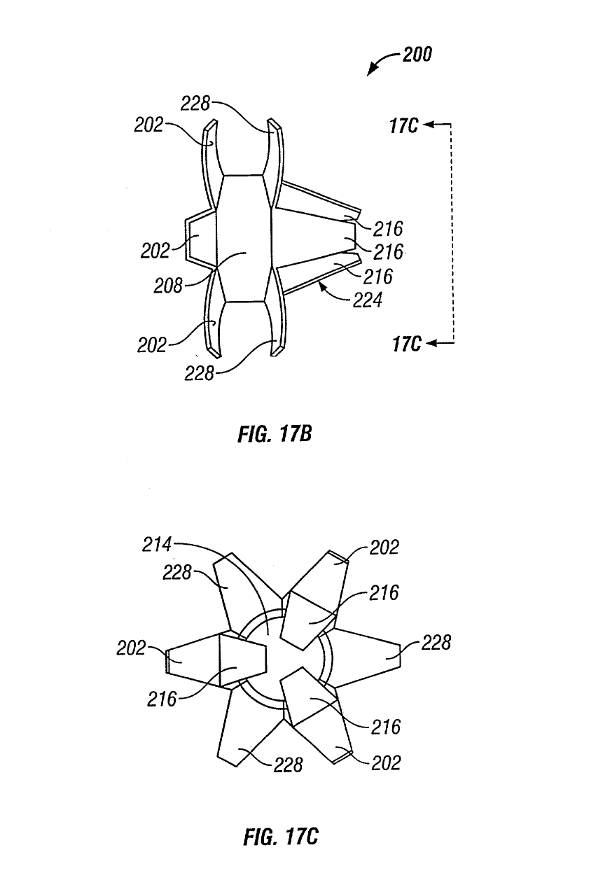

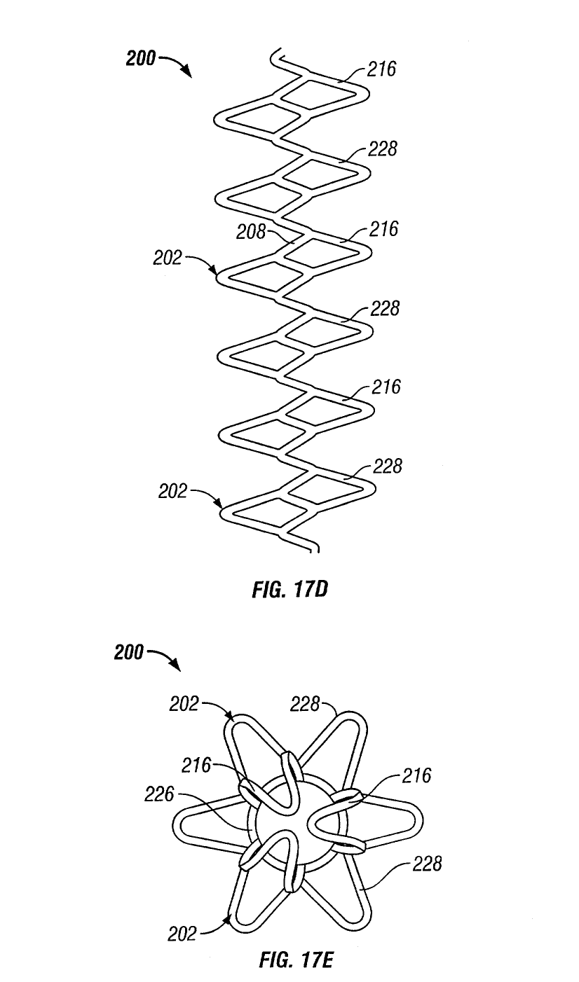

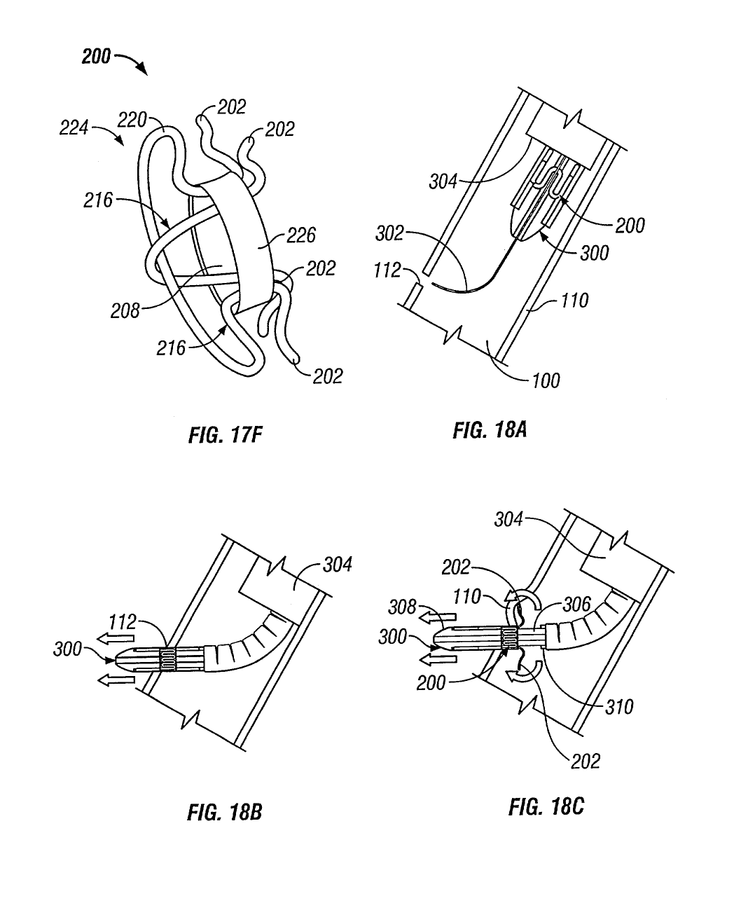

FIG. 17A illustrates another variation of a conduit 200. In this variation, the conduit 200 comprises a tube having a pattern which forms the extension members 202, 216, 228 and center section 208. FIG. 17A illustrates the conduit 200 in a pre-deployed configuration. FIG. 17B illustrates the conduit 200 after deployment. As illustrated, the proximal extension members 202 rotate about the center section 208. This variation illustrates a conduit 200 having both cage members 216 and distal extension members 228 on a distal end 212 of the center section 208. The distal extension members 228 are similar to the proximal extension members 202 in that they rotate about the distal end 212 of the center section. After rotation, proximal extension members 202 along with the distal extension members 228 assist in retaining the conduit 200 within the airway walls as tissue is located between the extension members 202, 228. The cage members 216 assume a cage structure 224 adjacent to a distal end of the passage of the conduit 200. As discussed above, the cage structure 224 formed by the cage members 216 provides a large area in the lung tissue to permit a larger volume of air to pass from the lung tissue into the conduit and also prevents parenchymal (or other) tissue from preventing air from passing through the conduit passage.

FIG. 17C illustrates a front view taken along the line 17C-17C of FIG. 17B. As illustrated, the cage members 216 move inwards to form the cage structure 224 over the passageway while the extension members 228 move in the opposite direction. In variations of the invention, the cage members 216 may move inwards sufficiently so that one or more of the cage members 216 touches. Similar to other conduits described herein, deployment of the conduits depicted in FIGS. 17A-17C may be accomplished, for example: via use of a pre-configured conduit that is restrained in a pre-deployment shape whereupon release of the restraints causes deployment; or a shape memory alloy capable of returning to a deployed shape upon reaching a certain temperature.

FIGS. 17D-17E illustrate a variation of the inventive conduit 200 similar to that shown in FIGS. 17A-17C. However, rather than being constructed from a tube structure, this variation comprises a frame, wire or mesh structure.

FIG. 17D illustrates an expanded planar view of the conduit 200 having both cage members 216 and distal extension members 228 on a distal end of the center section 208. The proximal end of the center section 208 includes a plurality of proximal extension member 202.

FIG. 17E illustrates a front view (from the distal to proximal end) of the conduit 200. As illustrated, the cage members 216 move inwards to form a cage structure 224 over the passageway while the extension members 228 move in the opposite direction. In variations of the invention, the cage members 216 may move inwards sufficiently so that one or more of the cage members 216 touches. As mentioned herein deployment of this conduit may be accomplished, for example: via use of a pre-configured conduit that is restrained in a pre-deployment shape whereupon release of the restraints causes deployment; or a shape memory alloy capable of returning to a deployed shape upon reaching a certain temperature. Moreover, as shown in FIG. 17E and for reasons described herein, the conduit 200 may also have a tissue barrier 226 located at the center section 208 of the conduit.

FIG. 17F illustrates another variation of a conduit 200. In this variation, the free ends of the cage members 216 are connected to form the cage structure 224 and the fixed ends of the cage members 216 extend to form the center section 208 of the conduit 200. The proximal extension members 202 are formed from the opposing end of the center section 208. The extension members and the center section of this variation of conduit may be formed from one or more wires or similar stock material. As illustrated, a tissue barrier 226 is placed over the center section 208. As discussed herein, the invention contemplates forming the tissue barrier 226 from an elastic material that expands upon deployment of the conduit 200.

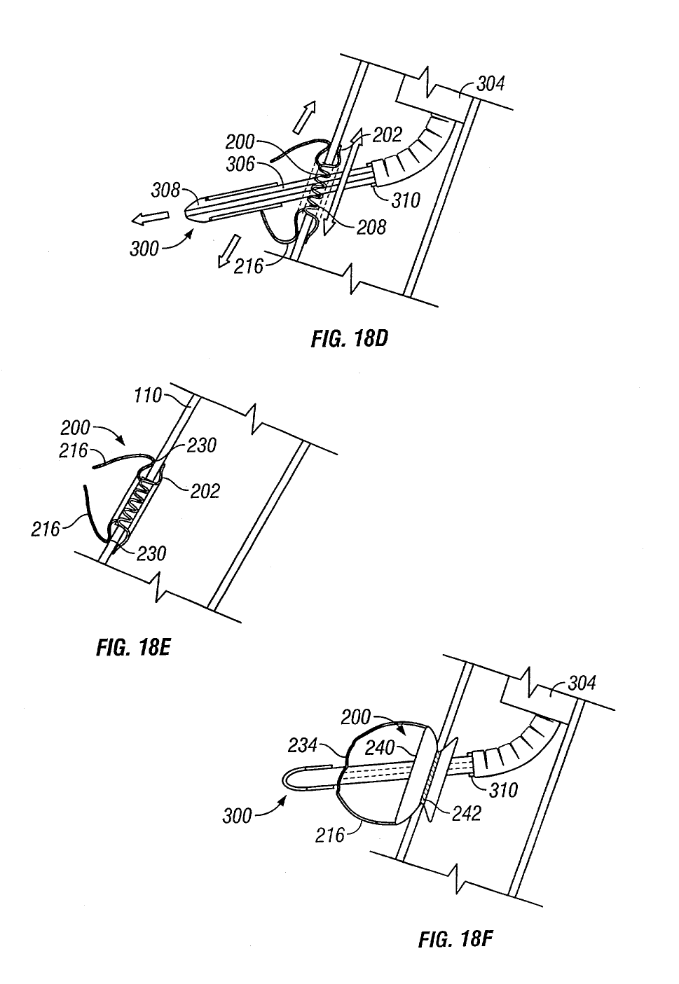

FIG. 18A illustrates a method for deployment of a conduit 200. First, a delivery device 300 is pre-loaded with a conduit 200. An access device 304 (e.g., an endoscope, a bronchoscope, or other device) may optionally be used to place the delivery device 300 adjacent to a collateral channel 112.

As the delivery device 300 approaches the collateral channel 112 a guide wire 302 may be used to place the delivery device 300 into the collateral channel 112. The guide wire 302 may be a conventional guide-wire or may simply be comprised of a super-elastic material. The use of a guide wire is optional as the invention contemplates placement of the conduit 200 using only the delivery device 300.

FIG. 18B illustrates the delivery device 300 after it is advanced through the collateral channel 112. As illustrated, if a guide wire 302 is used, it may be withdrawn from the site. FIG. 18B also illustrates articulation (or bending) of the deliver device 300 to access the collateral channel 112. However, the invention also contemplates that the access device 304 may be articulated to position the delivery device 300 such that it may advance through the collateral channel 112. In such a case, the delivery device 300 may exit straight from the access device 304. In such cases, the delivery device 300 may or may not articulate within the access device 304.

FIG. 18C illustrates deployment of the conduit 200. As illustrated, the conduit 200 may be pre-loaded on the delivery device 300 such that the conduit 200 is restrained about an inner shaft/catheter 306 by outer tubular members 308, 310. As described herein, the conduit 200 maybe comprised of an elastic or super-elastic material which is restrained in a reduced profile for deployment. Upon release of the restraints, the conduit 200 assumes a deployed shape. FIG. 18C shows a variation of the invention where the outer tubular members 308, 310 are in a telescoping arrangement. In such a case, the inner catheter 306 is advanced moving the first outer tubular member 308 and the conduit 200 distally relative to the second tubular member 310. As shown by the arrows, release of the proximal extension members 202 from the restraint causes the proximal extension members 202 to rotate about the center section of the conduit 200 to engage the airway wall 110.

FIG. 18D illustrates the next step as the inner catheter 306 and first outer tubular member 308 are again advanced. However, the proximal extension members 202 of the conduit 200 prevent the conduit 200 from advancing. The relative movement of the first outer tubular member 308 to the conduit 200 releases the cage members 216 of the conduit 200. As a result, the cage members 216 form a cage structure upon their release from the first outer tubular member 308. As illustrated in FIG. 18D, in some variations of the invention, once the conduit 200 is no longer restrained, the conduit's center section 208 may expand to assume a deployed state or profile.

FIG. 18E illustrates the deployed conduit 200 once the delivery device 300 is removed from the site. In this variation of the invention, the conduit 200 contains cage members 216 where the cage members include a portion 230 that is adapted to assume a position substantially transverse to the passage. Accordingly, this portion 230 of the cage member 216 functions to maintain the conduit 200 within the airway wall 110 in a manner similar to the proximal extension members 202.

FIG. 18F illustrates another deployed conduit having a cage 216, a tissue barrier 240 and a marker 242. The proximal extension members are covered by the tissue barrier. The conduit 200 shown in this figure may be deployed when the marker 242 is aligned with the channel opening. The marker is conspicuous and thus its position is readily ascertainable via an endoscope or other vision instrument.

Accordingly, the inventive conduit described herein may be formed of a plastically deformable material such that the conduit is expanded and plastically deforms into a deployed configuration. The conduit could also comprise a shape memory alloy that, upon reaching a particular temperature (e.g., 98.5° F.) assumes the deployed configuration. Moreover, the conduit may be constructed to have a deployed configuration, but is elastic such that it may be restrained in a pre-deployed configuration. As such, removal of the restraints causes the conduit to assume the deployed configuration. A conduit of this type could be, but is not limited to being, comprised from a shape memory alloy or a thermoplastic elastomer.

The conduits of this invention may be formed from a tube that is cut to form the extension members, center section, and cage portion. Alternatively, the conduit may be formed from a cylinder with the passageway being formed through the conduit. The conduit may also be formed from a sheet of material in which a specific pattern is cut. The cut sheet may then be rolled and formed into a tube.

The conduits described herein may be comprised of a metallic alloy material (e.g., stainless steel, 316 stainless steel, titanium, titanium alloy, MP35N, etc.), a shape memory alloy, a super-elastic alloy (e.g., a NiTi alloy), a shape memory polymer, a polymeric material, a material with rigid properties, a material with elastomeric properties, or a combination thereof. The conduit may be designed such that its natural state is an expanded state and it is restrained into a reduced profile, or, the conduit may be expanded into its expanded state by a variety of devices (e.g., a balloon catheter.) The conduit described herein may be manufactured by a variety of manufacturing processes including but not limited to laser cutting, chemical etching, punching, stamping, etc.

As mentioned above, the number of and cross sectional area of the extension members on a conduit may be selected as needed for the particular application. Also, the extension members may bend/pivot in such a way that they anchor into the tissue thereby securing placement of the conduit. Or, the extension members or the center section may contain barbs or other similar configurations to better adhere to the tissue. Moreover, the orientation of the extension members may vary as well. For example, the extension members may be configured to be radially expanding from the center section, or they may be angled with respect to a central axis of the conduit.

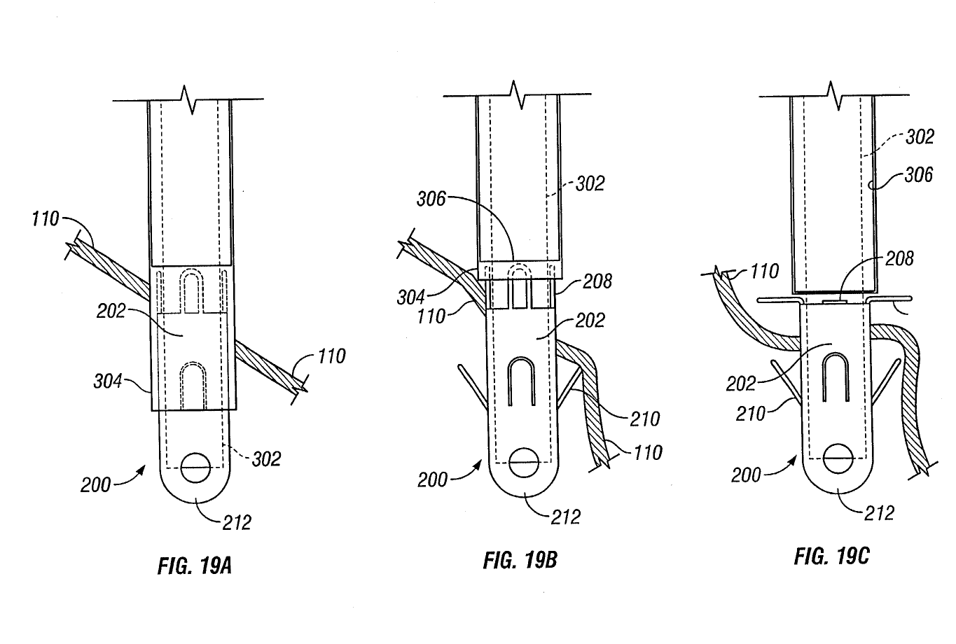

FIGS. 19A-19C illustrate deployment of a conduit 200. FIG. 19A illustrates a conduit placed on a delivery device 302, with an outer sheath 304 covering a portion of the conduit 200. The delivery device 302 and the outer sheath 304 direct the conduit 200 to a site of a collateral channel which, as described above, is an opening in an airway wall 110. An endoscope, bronchoscope, or similar device may be used to direct the delivery device, sheath, and conduit to the site. In such a case, the delivery device is usually advanced through the working channel of the bronchoscope/endoscope. Upon advancement of the conduit 200 to the site, the cage 212 end of the conduit 200 (this end also being referred to as the “parenchymal side”) is inserted through the airway wall 110 and into the parenchymal tissue of the lung. In most cases, the center section 202 of the conduit 200 remains in the collateral channel and is surrounded by the airway wall 110. As described elsewhere, the center section 202 of the conduit 200 may have a covering or other means to prevent tissue growth through the conduit 212 and into the conduit passage.

FIG. 19B illustrates withdrawal of the outer sheath 304 from the conduit 200 after the conduit 200 is suitably positioned in the tissue. As the outer sheath 304 is withdrawn, distal extension members 210 rotate about the center section 202 of the conduit 200. The distal extension members 210 may automatically deploy without external means. For example, the conduit 200 may be comprised of a shape memory alloy, or, the extension members may be resiliently biased to expand, etc. Alternatively, the distal extension members 210 may be rotated by means of a balloon, or similar actuation means, or application of current may cause the extension member 210 to assume the deployed position. In any case, once the distal extension members 210 deploy, the extension members prevent migration of the conduit 200 into the airway.

FIG. 19C illustrates deployment of the proximal extension members 208. Like the distal extension members 210, the proximal extension members 208 may deploy automatically, or they may be actively deployed. For example, FIG. 19C illustrates a proximal extension member 208 rotating about the center section 202 of the conduit 200. As shown, the proximal extension members 208 work in conjunction with the distal extension members 210 to retain the airway walls therebetween. As a result, the conduit is unable to migrate out of the airway wall 110. FIG. 19C also demonstrates how placement of the cage 212 or parenchymal end of the conduit 200 within the parenchyma provides a structure facilitating the improved release of trapped gasses from a hyper-inflated lung.

It is noted that the variation of the conduit depicted in FIGS. 19A-19C is rounded and closed at the distal end of the cage 212. As such, it is not necessary that the passage of the conduit 200 extends throughout the entire conduit 200. All that is required is that the passage extend through to the cage 212 of the conduit such that trapped air may pass through the cage 212 and along through the conduit.

It is further noted in FIGS. 19A-19C that the illustrated conduit 200 is a variation where the center section 202 of the conduit 200 includes openings once the distal extension members 210 rotate from the conduit 200. As discussed above and as illustrated in FIGS. 19B and 19C, the distal extension members 210 may rotate sufficiently such that the portion of the center section 202 with openings is positioned outside of the airway wall 110. However, the invention is not necessarily limited as such.

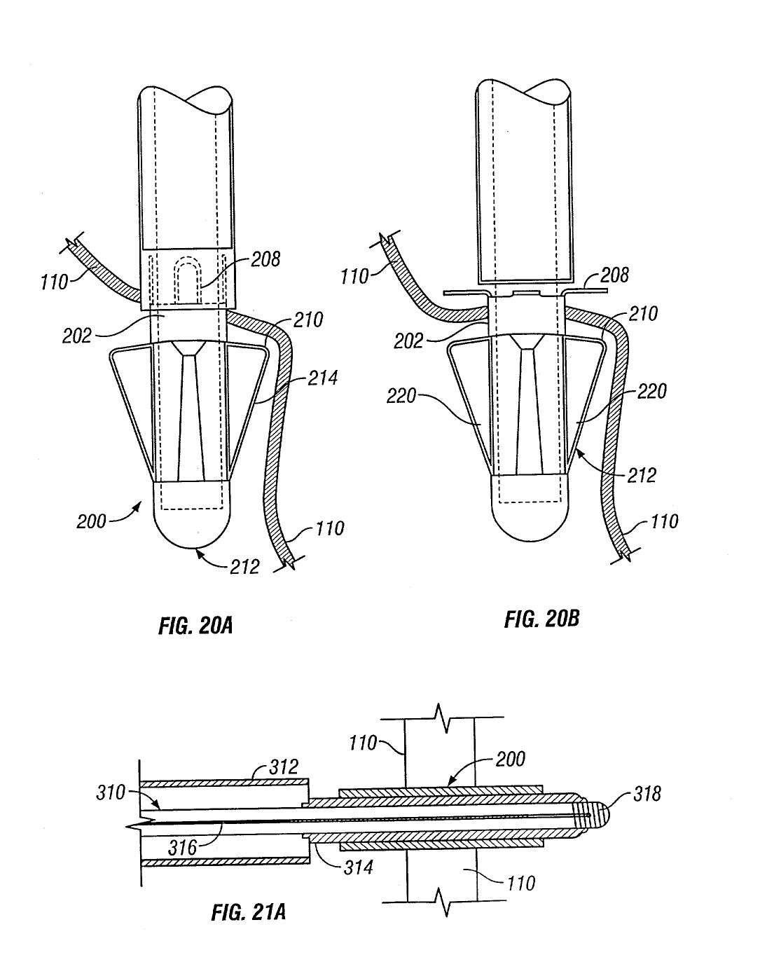

FIGS. 20A-20B illustrate deployment of another variation of a conduit 200. FIG. 20A illustrates the conduit 200 after placement within an airway wall 110 and after deployment of the distal extension members 210. As seen in FIG. 20A, the conduit 200 includes a cage 212 having cage members 214 which are attached to a free (or movable) end of the distal extension member 210. Accordingly, as the distal extension members 210 rotate about the center section 202 of the conduit 200, the extension members 210 also expand a portion of the cage 212 into an expanded state.

FIG. 20B illustrates the conduit of FIG. 20A after deployment of proximal extension members 208. As shown in FIG. 20B, the proximal and distal extensions 208, 210 prevent the conduit 200 from migrating from its deployment site within the airway wall 110. Furthermore, the expanded cage 212 having openings 220 therein, provides an expanded structure facilitating the improved release of trapped gasses from a hyper-inflated lung.

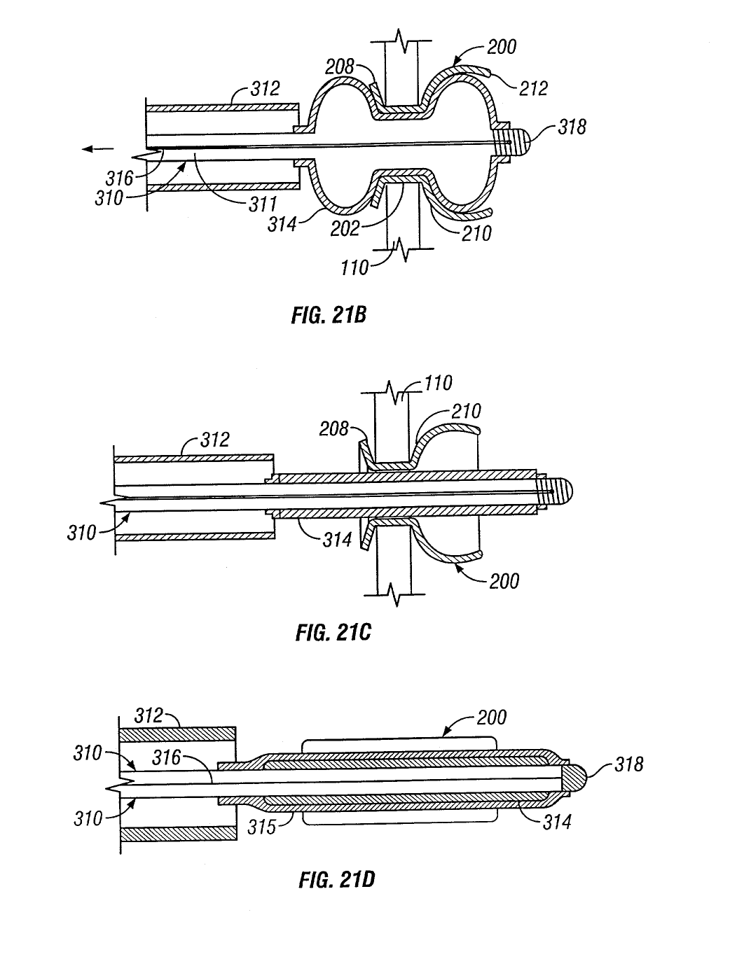

FIGS. 21A-21C illustrate another way to deploy a conduit 200 using a balloon catheter 310. The balloon catheter 310 will have an expandable balloon 314 at a distal end. The conduit 200 will be placed over the balloon section 314. The conduit 200 shown in FIGS. 21A-21C is for illustration purposes only. As such, features of the conduit (e.g., openings, outer coverings, etc.) are omitted for the sake of clarity. An outer sheath 312 may be used to advance the balloon catheter 310 and conduit 200 to the desired site within the airways. It is noted that the delivery device shown in FIG. 21A may also have another lumen which permits advancement of a guide wire (not shown) to assist in location of the collateral channel and placement of a conduit within the channel (see below). The balloon catheter 310 may also have an axial member 316 (e.g., wire, rod, etc.) that is fixedly attached to a distal end 318 of the balloon catheter 310. As indicated in the figure, the assembly will be advanced through an airway wall 110.

FIG. 21B illustrates deployment of the conduit 200. As illustrated, the balloon 314 includes first, second, and third portions where the first and third portions expand to a greater diameter than the second portion. This expansion of the balloon 314 forces the proximal and distal extension members 208, 210 to rotate about a center section 202 of the conduit 200. This variation of the conduit 200 also includes a cage 212 that achieves an expanded state as a result of inflation of the balloon 314. However, for conduit variations not having an expandable cage, a balloon may be adapted to deploy the proximal and distal extension members only. Although not illustrated, it is also contemplated that the inventive conduits may be deployed with a balloon catheter that includes one or more balloon portions that do not expand to different sizes. In such a case, the conduit may be adapted to assume a pre-designed deployed shape upon expansion of the balloon catheter. Accordingly, the pre-designed shape of the conduit rather than a balloon catheter may dictate the shape of the conduit after deployment.

FIG. 21B also illustrates the axial member 316 pulling the distal end 318 of the balloon catheter 310 in a proximal direction as indicated by the arrow. Such an action may further assist in the deployment of the conduit 200 as the balloon 314 compresses in an axial/longitudinal direction. The balloon catheter shaft 311 or the outer sheath 312 may assist in compression of the balloon 314 as the pulling of the distal end 318 against the balloon catheter shaft 311 or the outer sheath 312 further compresses the balloon 314.

When the conduit 200 is sufficiently deployed, the axial member 316 can be pushed to facilitate straightening or stretching of the balloon 314 in an axial/longitudinal direction which further eases removal of the balloon catheter 310 from the conduit 200.

FIG. 21C illustrates conduit 200 deployed about an airway wall 110. As discussed throughout this disclosure, the proximal and distal extension members 208, 210 retain the conduit 200 in the airway wall. Then, the balloon catheter 310 may be withdrawn into the outer sheath 312 and both the catheter 310 and sheath 312 are withdrawn from the site leaving the conduit 200 in place. The balloon 314 may serve several functions. As discussed above, the balloon 314 can expand to rotate the extension members 208, 210. Moreover, the balloon 314 may also serve to center the conduit 200 within the collateral channel, as it simultaneously begins to expand the conduit 200 thereby securing the conduit 200 within the collateral channel.

FIG. 21D illustrates another variation of a balloon having two layers of balloon material. The first balloon layer 314 provides increased burst strength against the pressurized fluid in the balloon and may or may not be elastomeric. The second balloon layer 315 may be elastomeric and may protect the first balloon from the edges of the conduit 200 during inflation of the balloon, and/or may assist in returning the first balloon layer 314 to a compressed state when the first balloon layer 314 deflates to ease removal of the catheter from the conduit 200.

The design of the balloon may be designed as needed to impart the desired angle to the extension members of the conduit. The balloons described herein may be constructed of polyethylene terephthalate (PET), nylon, or another material which is used in constructing inflatable members such as the balloon members used in balloon catheters.

It should be noted that deployment of conduits of the present invention is not limited to that shown above, instead, other means may be used to deploy the conduits. Other means for deployment of the conduits of the present invention include, but are not limited to, mechanical wedges, lever-type devices, scissors-jack devices, open chest surgical placement, etc.

A balloon catheter can be used to deploy conduits made from materials that lack shape memory properties. That is to say, in some cases, a balloon catheter can deploy conduits that are inelastic or plastically deformable. Additionally, when using a balloon catheter to deploy the conduit, the balloon member itself may be made of an elastic or inelastic material. The balloon member may also have various shapes or profiles including but not limited to cylindrical, spherical and hourglass shapes. The balloon may be elastic or inelastic. An inelastic cylindrical balloon, for example, can be used to deploy conduits which have diametrically or otherwise controlled shapes. Again, as described above, the conduits of the present invention may include control segments which limit the shape of the conduit to a maximum or certain deployed shape. However, in conduits that lack control members or in conduits that are otherwise not inherently shape restricted, the balloon member can impose its shape onto the conduit when the balloon member is expanded. For example, an hourglass-shaped balloon may impose an hourglass shape onto a shapeable conduit thereby securing the conduit in a channel.

It is contemplated that conduits may be attached to a delivery device using the natural resiliency of the conduit, or, in those cases where the conduit is spring loaded, the conduit may be restrained in a reduced profile and may be removably affixed to the delivery device using an adhesive, or a removable sleeve such as a heat shrink tube. In such cases, expansion of the conduit using, for example, balloon causes release of the conduit by release of the adhesive or breaking of the heat shrink tubing. The means of attachment may be bioabsorbable and remain in the body, or may remain affixed to a delivery device and is removed upon removal of the delivery device.

Another method of deploying a conduit includes restraining the conduit about a delivery device using a wire or string tied in a slip-knot or a series of slip-knots. When the conduit is delivered to a desired location, the proximal end of the wire or string may be pulled which releases the wire/string and deploys the conduit.

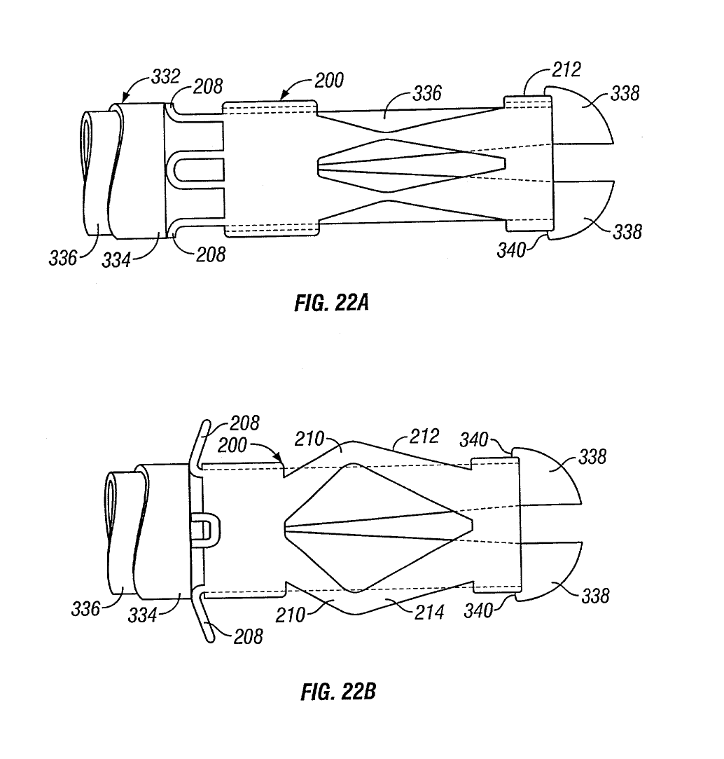

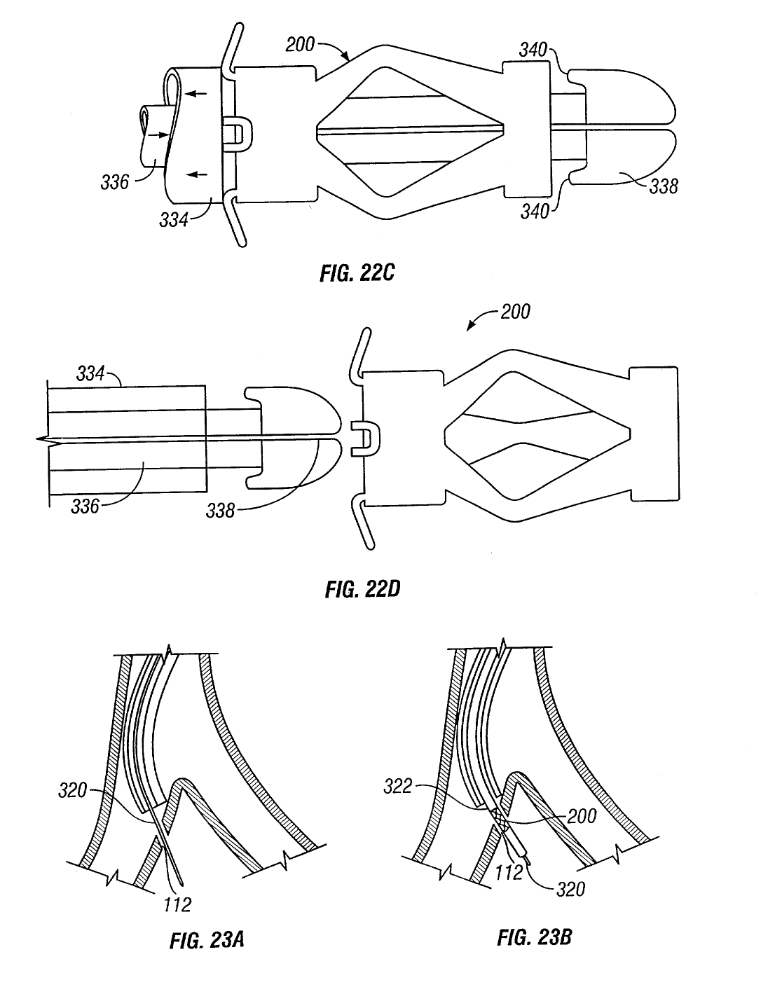

FIGS. 22A-22D illustrate a method for deploying a conduit 200. As illustrated in FIG. 22A, a conduit 200 is placed onto a deployment catheter 332. The deployment catheter 332 may comprise a sheath 334 and a shaft 336. The shaft 336 may contain cap portions 338. In such a case, prior to deployment, the conduit 200 may be loaded on the shaft 336 between the sheath 334 and cap portions 338. For example, the proximal extension members 208 may be placed adjacent to the sheath 334 while the cage 212 is placed against the cap portions 338. In some variations of the invention, one or more of the cap portions 338 may have a lip 340 which retains the conduit cage 212. It is contemplated that a conduit 200 may be delivered to a user pre-loaded on the catheter 332 in such a pre-deployed configuration.

FIG. 22B illustrates expansion of the cage 212 by moving shaft 336 relative to the sheath 334 as shown by the arrow. In this way, the conduit 200 compresses between the sheath 334 and the cap portions 338. Compression of the conduit 200 causes the proximal and distal extension members 208, 210 to rotate and move away from the shaft 336. In the conduit 200 variation depicted in FIG. 22B, the conduit 200 includes cage members 214 which follow the free end of the distal extension member 210 and move away from the shaft 336 to form the expanded cage.

Moreover, the invention may include a deployment catheter that is configured to be easily removed from the deployed conduit 200. For example, the cap portions 338 of the shaft 336 may be spring biased to remain together. In such a case the cap portions 338 of the shaft 336 will have a natural state where the cap portions 338 remain immediately adjacent to one another. Given this configuration, when the cap portions 338 are attached to the conduit 200, as shown in FIG. 22B, the conduit 200 prevents the cap portions 338 from returning to their natural state. As discussed above, the cap portions 338 may have a lip 340 which assists securing the cap portions 338 on the conduit 200. As shown in FIG. 22C, movement of the shaft 336 in a distal direction (as indicated by the arrows), permits disengagement of the lip 340 of the cap portions 338 from the conduit 200. This action permits the cap portions 338 to close together.

As illustrated in FIG. 22D, once the cap portions 338 return to their natural state, the conduit 200 and shaft 336 may be de-coupled. Decoupling of the conduit 200 and shaft 336 may be achieved by, for example, withdrawal of the shaft 336 from the conduit, or, use of the sheath 334 to advance the conduit 200 over the shaft 336 and cap portions 338.

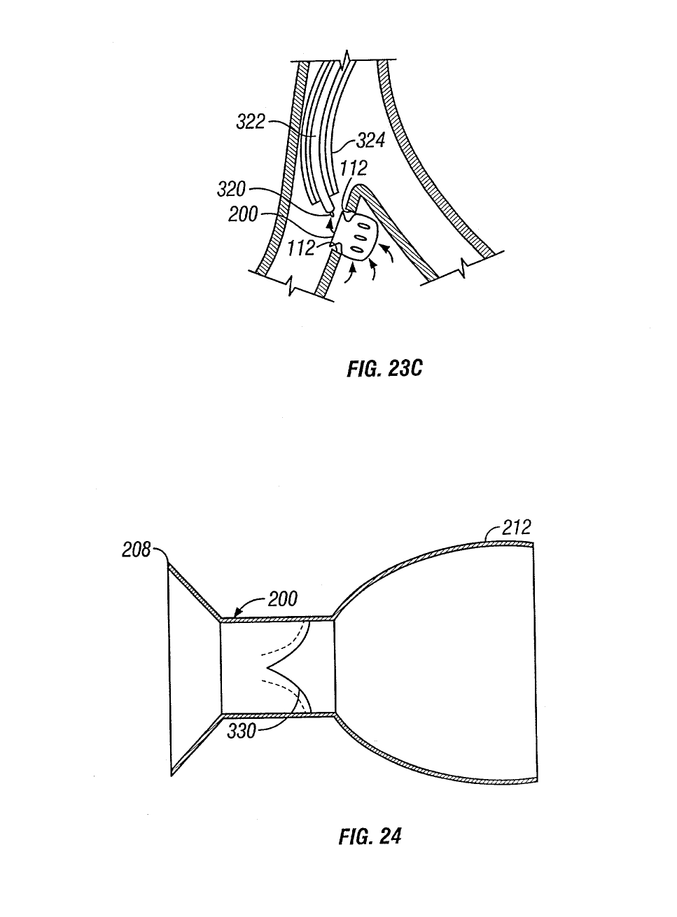

FIGS. 23A-23C illustrate placement of a conduit via use of a guide-member. FIG. 23A illustrates directing a guide-member, such as a guide-wire 320, or other similar device into a collateral channel 112.

FIG. 23B illustrates the advancement of a catheter device 322 into the collateral channel 112. The catheter 322 may advance over the guide-member 320 and into the collateral channel 112. As discussed above, the conduit 200 is then deployed in the collateral channel 112.

FIG. 23C illustrates the conduit 200 deployed within the collateral channel 112 and the withdrawal of the guide-member 320, catheter 322, and access device 324 (e.g., a bronchoscope, endoscope, or similar device). As shown by the arrows of FIG. 23C, the conduit 200 maintains the collateral channel 112 so that trapped non-functional air is evacuated from a hyper-inflated lung.

It is noted that a variation of the inventive method includes using a guide-wire to create the collateral channel, and leaving the guide-wire to extend through the collateral channel. Accordingly, a conduit may be advanced over the guide-wire into the collateral channel.

The invention also contemplates the use of conduits to deliver drugs or medicines to the area of the collateral opening. Also contemplated is the use of a fibrin, cyano-acrylate, or any other bio-compatible adhesive with the conduit to maintain the patency of the collateral channel. For example, the adhesive could be deposited on the exterior of the conduit to maintain patency of the channel. Also, the use of a bioabsorbable material may promote the growth of epithelium on the walls of the conduit. For example, covering the exterior of the conduit with small intestine submucosa, or other bioabsorbable material, may promote epithelium growth thus securing the conduit while the bioabsorbable material eventually absorbs into the body.

FIG. 24 illustrates a variation of a conduit 200 having a one-way valve 330. The valve 330 allows the conduit 200 to permit exhaust of gas from the air sac but prevents the conduit 200 from serving as another entrance of gas to the air sac. The valve 330 may be placed at ends of the conduit or within a lumen of the conduit. When such a conduit is deployed in a collateral channel, air may flow into the cage 212, through the valve 330, and out the proximal portion 208 of the conduit into an airway thereby releasing excess trapped air from a lung. The valve 330 may also be used as bacterial in-flow protection for the lungs. A conduit having a valve may be placed in a channel in an airway wall so that air may flow from the parenchymal tissue into the airway and out the lung.

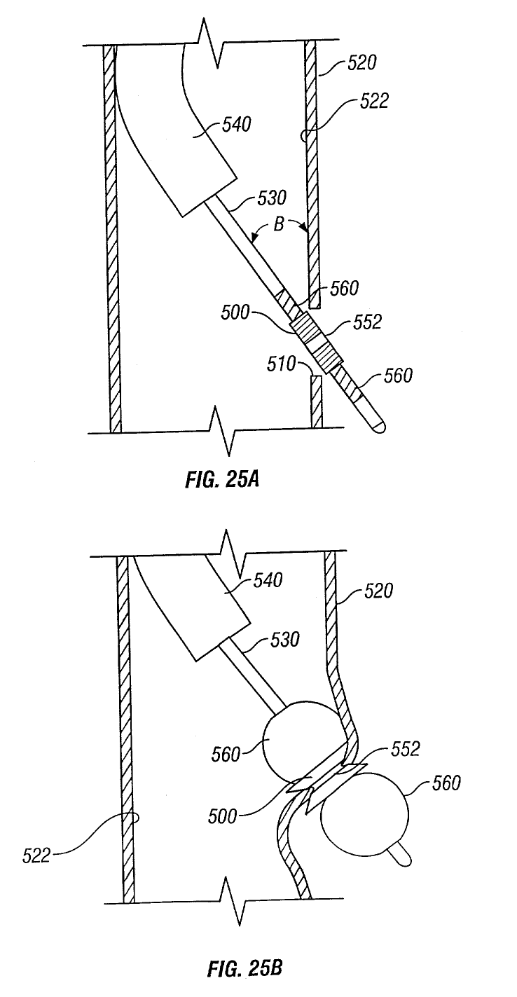

FIGS. 25A-25B illustrate another example of deploying a conduit 500 in a channel 510 (or opening) created in a tissue wall 520. Referring to FIG. 25A, a delivery tool 530 carrying a deployable conduit 500 is inserted into the channel 510. The conduit 500 may include a distal cage structure. The delivery tool 530 is extended straight from an access catheter 540 such that the delivery tool forms an angle β with the tissue wall 520. It is to be understood that while the tissue wall of airway 522 is shown as being thin and well defined, the present invention may be utilized to maintain the patency of channels and openings which have less well defined boundaries. The delivery tool is further manipulated until the conduit is properly positioned which is determined by, for example, observing the position of a visualization mark 552 on the conduit relative to the opening of the channel 510.

FIG. 25B illustrates enlarging and securing the conduit in the channel using an expandable member or balloon 560. The balloon 560 may be radially expanded using fluid (gas or liquid) pressure to deploy the conduit 500. The balloon may have a cylindrical shape (or another shape such as an hourglass shape) when expanded to 1.) expand the center section and 2.) deflect the proximal and distal sections of the conduit such that the conduit is secured to the tissue wall 520. During this deployment step, the tissue wall 520 may distort or bend to some degree but when the delivery tool is removed, the elasticity of the tissue tends to return the tissue wall to its initial shape. The delivery tool tends to center the conduit in the channel even when deployed at an angle. Also, it should be noted that despite the inflatable member's cylindrical shape, the conduit is deployed in the proper shape since certain portions of the conduit (e.g., the center section and or cage) are diametrically restricted using control segments. Accordingly, the conduits disclosed herein may be deployed in controlled shapes and inserted into the tissue wall at either a perpendicular (or non-perpendicular) angle.

A medical kit for improving gaseous flow within a diseased lung may include a conduit, a hole-making device, a deployment device and/or a detection device. Examples of such methods and devices are described in U.S. patent application Ser. No. 09/633,651, filed on Aug. 7, 2000; U.S. patent application Ser. Nos. 09/947,144, 09/946,706, and 09/947,126 all filed on Sep. 4, 2001; and U.S. patent application Ser. Nos. 10/080,344 and 10/079,605 both filed on Feb. 21, 2002, each of which is incorporated by reference in its entirety. The kit may further contain a power supply, such as an RF generator, or a Doppler controller which generates and analyzes the signals used in the detection devices. The kit may include these components either singly or in combination.

The kit of the present invention may also contain instructions teaching the use of any device of the present invention, or teaching any of the methods of the present invention. The instructions may actually be physically provided in the kit, or it may be on the covering, e.g., lidstock, of the kit. Furthermore, the kit may also comprise a bronchoscope, or guide-member (such as a guide-wire), or other such device facilitating performance of any of the inventive procedures described herein. All the components of the kit may be provided sterile and in a sterile container such as a pouch or tray. Sterile barriers are desirable to minimize the chances of contamination prior to use.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

All of the features disclosed in the specification (including any accompanying claims, abstract and drawings), and/or all of the steps of any method or process disclosed, may be combined in any combination, except combinations where at least some of such features and/or steps are mutually exclusive.

1. A conduit comprising:
a center section comprising a first end, a second end and a center-section passageway extending from said first end to said second end;
a plurality of first extension members extending from said first end, said first extension members being deflectable about said first end of said center section; and
a cage structure adjacent to said second end, said cage structure having at least one opening and at least one cage passageway in fluid communication with said center-section passageway.

2. The conduit of claim 1 wherein said conduit comprises a wire frame.

3. The conduit of claim 1 wherein said cage structure is connected to said second end of said center section by a plurality of second extension members, said second extension members being outwardly deflectable about said second end of said center section.

4. The conduit of claim 1 wherein said cage structure is connected directly to said second end of said center section.

5. The conduit of claim 1 further comprising a tissue barrier coaxially surrounding at least a portion of said center section.

6. The conduit of claim 5 wherein said tissue barrier at least partially surrounds said center section and at least partially surrounds said cage structure.

7. The conduit of claim 6 further comprising a visualization mark, said visualization mark being visually distinct from another portion of said conduit.

8. The conduit of claim 7 wherein said visualization mark is an opaque ring.

9. The conduit of claim 7 wherein said visualization mark is a white ring.

10. The conduit of claim 9 wherein said visualization mark comprises titanium dioxide.

11. The conduit of claim 5 further comprising a bioactive substance on said tissue barrier.

12. The conduit of claim 1 wherein said conduit is radially expandable.

13. The conduit of claim 12 wherein said cage structure has an unexpanded diameter and an expanded diameter at a point along the cage passageway and wherein a ratio of said unexpanded diameter to said expanded diameter ranges from 1:2 to 1:4.

14. The conduit of claim 1 wherein said cage structure comprises a plurality of elongate cage members.

15. The conduit of claim 14 wherein at least one of said cage members has an axially extendable length.

16. The conduit of claim 15 wherein said at least one extendable cage member has a zigzag region which may be straightened.

17. The conduit of claim 2 wherein said wire frame structure is plastically deformable.

18. The conduit of claim 2 wherein said first extension members outwardly deflect when the first extension members are not restrained.

19. The conduit of claim 1 wherein each of said first extension members comprises a fixed end and a movable tapered end, said movable tapered end having a width less than the width of said first extension member at said fixed end.

20. The conduit of claim 14 wherein each of said elongate cage members comprises a tapered end.

21. The conduit of claim 14 wherein said cage members form a tubular shape.

22. The conduit of claim 3 wherein said cage members from a tubular shape.

23. The conduit of claim 4 wherein said cage structure is comprised of a plurality of elongate cage members and wherein each of said cage members has a fixed end attached to said second end of said center section, an intermediate section, and a free end distal to said intermediate section wherein a portion of each said cage member between said intermediate section and said fixed end may outwardly deflect such that said portion is substantially transverse to said center section passage.

24. The conduit of claim 14 further comprising cage-control segments that link adjacent cage members and limit radial expansion of said cage structure.

25. The conduit of claim 1 wherein each of said first extension members comprises a fixed end and a free end, said fixed end attached to said first end of said center section at a weakened region of reduced cross sectional area.

26. The conduit of claim 25 wherein said weakened region has a reduced thickness.

27. The conduit of claim 12 wherein said conduit is adapted to axially shrink while radially expanding.

28. The conduit of claim 5 wherein said tissue barrier comprises a material selected from the group consisting of silicone and urethane.

29. The conduit of claim 2 wherein said wire frame comprises a material selected from the group consisting of stainless steel, titanium, titanium alloy, nitinol, and MP35N.

30. The conduit of claim 1 wherein said cage structure has an axial length ranging from 2 to 20 mm.

31. The conduit of claim 12 further comprising at least one center-control segment configured to restrict radial expansion of said center-section passageway to a maximum diameter.

32. The conduit of claim 5 wherein said tissue barrier coaxially surrounds said proximal extension members and wherein said conduit further comprises at least one membrane-tearing support extending from one of said first extension members to an adjacent first extension member such that when said first extension members are outwardly deflected, said at least one membrane-tearing support straightens and holds the tissue barrier at a point between adjacent outwardly deflected first extension members.

33. The conduit of claim 6 wherein said cage structure comprises a plurality of elongate cage members and said tissue barrier coaxially surrounds said cage structure and wherein said conduit further comprises at least one membrane-tearing support extending from one of said cage members to an adjacent cage member such that when said cage members are outwardly deflected, said at least one membrane-tearing support straightens and holds the tissue barrier at a point between adjacent deployed cage members.

34. The conduit of claim 1 wherein said at least one opening is at said end of said cage structure.

35. The conduit of claim 1 wherein said at least one opening is in a side wall of said cage structure.

36. The conduit of claim 2 further comprising a bioactive substance on said wire frame.

37. A conduit having an undeployed state for facilitating delivery to a channel in a lung and a deployed state, different than the undeployed state, for maintaining the patency of the channel, said conduit comprising:
a radially expandable frame having a proximal section, a center section and a distal section, said proximal section comprising a plurality of proximal extension members, said center section comprising a first end at which said plurality of proximal extension members are attached, a second end, and a center-section passage extending from said first end to said second end, said distal section comprising a cage distal to said center section and said cage comprising at least one opening and at least one cage passage which is in fluid communication with said center-section passage wherein when said conduit is in said undeployed state, said proximal section, said center section and said distal section have a reduced profile, and when said conduit is in said deployed state, said plurality of extension members deflect outward forming a non-zero angle with an axis of said center-section passage, and said cage has an expanded profile greater than that of said cage when said conduit is in said undeployed state.

38. The conduit of claim 37 wherein each of said proximal extension members comprises at least one aperture.

39. The conduit of claim 37 wherein said cage is connected to said second end of said center section by a plurality of second extension members, said second extension members being deflectable about said second end of said center section.

40. The conduit of claim 37 wherein said cage comprises a plurality of cage members that are directly connected with said second end of said center section, each of said cage members having a fixed end, a movable end, and an intermediate segment therebetween, said fixed end being attached to said center section second end, and wherein said intermediate segment and said movable end are adapted to assume a cage shape when not constrained.

41. The conduit of claim 37 wherein said cage in said expanded profile has a varying diameter.

42. The conduit of claim 37 wherein said conduit in said undeployed state has a constant diameter.

43. The conduit of claim 41 wherein said cage has a first end and a second end and said diameter is greater in a middle portion between said first end and said second end.

44. The conduit of claim 37 wherein each of said proximal extension members comprises a weakened portion of reduced cross section such that each of said proximal extension members bends at said weakened portion when a radially outward force is applied to said proximal extension members.

45. The conduit of claim 37 wherein said center section is radially expandable and said center section has a greater diameter in said deployed state than in said undeployed state.

46. The conduit of claim 45 further comprising at least one center-control segment configured to restrict radial expansion of said center-section passageway to a maximum diameter.

47. The conduit of claim 46 wherein said at least one center-control member has an arcuate shape when said center section is not radially expanded.

48. The conduit of claim 47 wherein said center section has a plurality of ribs and said center control segment joins two adjacent ribs.

49. The conduit of claim 37 wherein said cage has an axial length ranging from 2 to 20 mm when deployed.

50. The conduit of claim 37 further comprising a biocompatible coating coaxially surrounding at least a portion of said frame.

51. The conduit of clam 39 wherein said proximal extension members and said distal extension members, when said conduit is in said deployed state, are deflected from said axis of said center-section passage such that tissue may be compressed between opposing proximal and distal extension members when said conduit is deployed in a channel.

52. The conduit of claim 51 wherein said proximal and distal extension members are configured such that, when said conduit is in said deployed state, said opposing proximal and distal extension members deflect to a degree such that a V-shape is formed when viewed from a side view.

53. The conduit of claim 37 wherein said cage passage, when deployed, has a greater diameter than that of said center-section passage, when deployed.

54. The conduit of claim 37 wherein said cage passage, when deployed, has a varying diameter from a first end of said cage passage to a second end of said cage passage, when deployed.

55. The conduit of claim 50 further comprising a bioactive substance disposed on at least a portion an outer surface of said conduit.

56. A conduit for maintaining the patency of a channel created in lung tissue, said conduit comprising:
a center section having a proximal end and a distal end and a center section passage within said center section extending between said ends;
a plurality of extension members comprising at least one proximal extension member and at least one distal extension member, said at least one proximal extension member having a fixed end attached to said proximal end of said center section and said at least one distal extension member having a fixed end attached to said distal end of said center section, each of said proximal and distal extension members having a free end being moveable such that said extension members may rotate about each of said ends of said center section to retain said tissue between said extension members; and
a cage adjacent to said distal end of said center section, said cage having at least one opening and a cage passage in fluid communication with said center section passage.

57. The conduit of claim 56 wherein said cage comprises a plurality of elongate members.

58. The conduit of claim 56 wherein said cage comprises a tubular body comprising a plurality of apertures in said body of said cage, each of said apertures in fluid communication with said cage passage.

59. A method for maintaining the patency of a channel in lung tissue comprising:
deploying a medical device in said channel said medical device having a passageway extending from one open end to a second open end.

60. The method of claim 59 wherein said medical device is a conduit as recited in claim 1.

61. The method of claim 60 wherein deploying is performed with a balloon catheter having an inflatable member.

62. The method of claim 60 further comprising delivering a bioactive substance to the tissue.

63. The method of claim 62 wherein the bioactive substance is a coating on the conduit.

64. The method of claim 62 wherein the substance is delivered by a delivery catheter prior to deploying the conduit.

65. The method of claim 61 wherein said balloon catheter comprises an opaque band coaxially surrounding a region of said inflatable member.

66. The method of claim 59 wherein said channel is a hole through an airway wall.

67. A kit comprising:
a conduit as recited in claim 1; and
a deployment catheter to deploy the conduit.

68. The kit as recited in claim 67 further comprising a guidewire.

69. The kit as recited in claim 67 wherein said deployment catheter is a balloon catheter.

70. The kit as recited in claim 67 further comprising an instrument for creating holes in an airway wall.
